(19) United States
(12) Patent Application Publication
(10) Pub. No.: US 2005/0086010 A1
(43) Pub. Date: Apr. 21, 2005

(54) Stochastic variable selection method for model selection

(75) Inventors: Jonnagadda Sunil Rao, Richmond Heights, OH (US); Hemant Ishwaran, Shaker Heights, OH (US)

Correspondence Address: FISH & NEAVE IP GROUP;ROPES & GRAY LLP, ONE INTERNATIONAL PLACE, BOSTON, MA, 02110-2624, US

(73) Assignees: Case Western Reserve University, Cleveland, OH (US); The Cleveland Clinic Foundation, Cleveland, OH (US)

(21) Appl. No.: 10/858,867

(22) Filed: Jun. 1, 2004

Related U.S. Application Data
(60) Provisional application No. 60/474,456, filed on May 30, 2003.

(51) Int. Cl. G06F 19/00; G01N 33/48; G01N 33/50

(52) U.S. Cl. 702/20

(57) ABSTRACT

A method of identifying differentially-expressed genes includes deriving an analysis of variance (ANOVA) or analysis of covariance (ANCOVA) model for expression data associated with a number of genes; from the ANOVA or ANCOVA model, deriving a linear regression model defined at least in part by an observation vector representative of an observed subset of the gene-expression data, a design matrix of regressor variables, a vector of regression coefficients representing gene contribution to the observation vector, and a measurement error vector; and to the linear regression model, applying a hierarchical selection algorithm to designate a subset of the regression coefficients as significant regression coefficients, the selection algorithm representing at least one of the observation vector, the design matrix, and the measurement error vector as being hierarchically dependent on parameters having predetermined probabilistic properties, wherein the designated subset corresponds to a respective subset of the genes identified as differentially expressed.

### CROSS-REFERENCE TO RELATED APPLICATIONS

This application incorporates by reference in entirety, and claims priority to and benefit of, U.S. Provisional Patent Application No. 60/474,456, filed on 30 May 2003.

### STATEMENT REGARDING FEDERAL FUNDING

Work described herein was funded, in part, by the National Institutes of Health, Grant No. K25-CA89867. The United States government has certain rights in the invention.

### BACKGROUND

The systems and methods described herein pertain to statistical data analysis in general, and to model selection in particular.

Multiple hypothesis testing—also known by model selection, data classification, multiple detection, and other descriptors—has become important in an age of increasingly sophisticated data-gathering technologies. The high data-throughput nature of instruments at our disposal not only creates an enormous wealth of information, but also poses a data-analysis challenge, because of the large multiple testing or classification problems involved.

A commonly-used approach has been to focus on one type of model selection error at the expense of other types. A drawback of such a method is that more subtle features of the data are missed, features that might give analysts more insight into how to test various hypotheses on collected data.

### SUMMARY OF THE INVENTION

It is desirable to devise a new method for performing model selection, data classification, significance testing, or hypothesis testing—especially in high-dimensional or large-scale data contexts: a new method that strikes a balance between tolerances for various error types.

In one aspect, the invention is a method of analyzing data, seeking to discern structure within the data that would then allow it to fit one or more possible models or hypotheses. Data being analyzed can be, among others things: mass spectroscopy data; gene expression data; protein expression data; genotyping data; metabolite production data; virus data; other types of biomolecular data; data collected from a large-scale—and possibly intractable—system, network or array; or data collected from any system wherein a type of classification decision has to be made based on, among other things, information contained in the data, or possibly even based on external information.

Accordingly, the methods and systems described herein can access and process data by casting an analysis or classification problem into, for example, the context and language of analysis of variance (ANOVA) or analysis of covariance (ANCOVA). An ANOVA or ANCOVA formulation for data analysis may be recast as a regression problem comprising an observation vector, a design matrix comprising a plurality of regressors, a vector of regression coefficients that are representative of the model, and a measurement error vector.

According to one practice, the systems and methods described herein apply a hierarchical model selection algorithm to classify the regression coefficients (which can serve as model parameters), into any number of groups, where the number of groups depends on the context. According to one exemplary embodiment, the classification, or model selection, problem involves only two groups. For such an embodiment, the selection algorithm seeks to identify the subset of the model parameters (equivalently, the subset of the regression coefficients) that are “significant.” This is also known as binary hypothesis testing, consisting of a null hypothesis and an alternative hypothesis. An example of this occurs in the analysis of differential gene expression data, wherein data is collected from two samples of gene arrays—one serving as a control sample and the other a treatment sample. The control sample may represent healthy tissue, while the treatment sample may be, for example, representative of cancerous tissue. In this embodiment, a goal then is to identify the subset of the genes that express themselves differently (alternative hypothesis) in the two samples, thus giving the investigator a clue as to which genes are “significant” in the particular cancer represented by the treatment group. The null hypothesis in this context would be that a gene does not differentially express itself.

The systems and methods described herein also apply, in various embodiments, to data analysis contexts involving more than two groups; a goal therein includes classifying the model parameters into one of a plurality of possible models or groups. In a multigroup embodiment the groups indicate, for example, stages of disease, and interest lies in finding genes that differentially express in various patterns across the stages. For example, genes that differentially express throughout a disease process such as cancer might be termed tumor progression genes and those that differentially express primarily during the early or primarily during the late stages may be termed early and late hit-and-run genes, respectively. In this setup, a particular stage (typically the most benign) acts as a baseline or reference from which stagewise differential gene expression is ascertained.

It is commonly known that statistical methods applied to classification problems involve various types of errors. In the case of two-way classification, for example, there are, among others, two types of errors: false discovery (otherwise known as false alarm) and false non-discovery (also known as a “miss” in certain contexts). The systems and methods described herein provide a mechanism by which a balance can be struck between tolerance for one type of error versus the other.

Prior art methods have focused primarily on keeping false discovery rates (that is, false alarm rates) in check. In the context of differential gene expression, this has led to falsely dismissing some genes as differentially inexpressive, even though they do contribute to the treatment sample.

The systems and methods described herein, however, strike a balance between two types of errors: false discovery and false non-discovery. By identifying a subset of the data having intermediate features that are less readily classifiable than other data points that have distinctly prominent features, the methods and systems described herein flag the analyst to pay closer attention to the intermediate data points, and use greater care in classifying them. In an embodiment involving differential gene expression data, for example, there are some genes whose expression levels are not as prominent, and hence they are not as clearly identifiable or classifiable, as others with distinctly prominent differential gene expression features. These intermediate genes sometimes do associate with the treatment sample; yet, they are missed by current methods, which focus primarily on false discovery rates.

The systems and methods described herein remedy the problem by applying a hierarchical selection algorithm to the ANOVA- or ANCOVA-based regression formulation of the data, to classify the regression coefficients into the number of groups under consideration in any given context. The selection algorithm defines a hierarchy of variables (referred to also as hyperparameters) with appropriately chosen probabilistic properties, to represent the regression coefficients, the observation vector, the design matrix, the measurement error vector, or any combination thereof. The probabilistic properties of the hyperparameters are chosen in a way so as to properly model the number of classification groups in the context of interest.

The systems and methods described herein further may involve a Bayesian approach, for example in the selection algorithm. One example of a hierarchical Bayesian approach is the parametric stochastic variable selection (P-SVS) algorithm.

The systems and methods described herein also may employ statistical techniques, such as appropriate scaling or mean subtraction, to decorrelate model parameters or simplify their respective, effective probabilistic complexities.

Posterior statistics of the model parameters are used by the systems and methods described herein, to define test statistics that may be used to perform the classification of data. For example, from the posterior density, the systems and methods disclosed herein may use the posterior mean of the regression coefficients (model parameters), conditioned on the observation vector, to test for significance of the model parameters (in the case two hypotheses) or to perform multi-group classification. In an alternative embodiment, the conditional median can be used as a test statistic.

The systems and methods described herein highlight how the posterior mean vector of the model parameters is simply a ridge estimate from a generalized ridge regression of the observation vector on the design matrix. Furthermore, the posterior mean may comprise a weighted average of shrunken posterior estimates of the model parameters.

Another distinctive feature of the systems and methods described herein is a graphical method that facilitates the proper identification and/or classification of the intermediate values of the data. By plotting the posterior variance against the posterior mean of the model parameters, the systems and methods disclosed herein highlight a feature of the intermediate data points that may otherwise be missed by other methods known in the art. Those of ordinary skill in the art know, or can readily ascertain having read this specification, that variations of the plot just described are possible; for example, the posterior mean may be replaced by the posterior median. Moreover, a generalization exists of the two-group BAM shrinkage plot extended to multiple groups. In one embodiment, a particular group is designated as a baseline, and individual two dimensional plots of pairwise BAM test statistic values (also called Bayes test statistics)-which are group effect test statistics against the designated baseline group-are plotted. According to one practice, a number of two-dimensionals plots are generated based, at least in part, on the total number of groups under study. Such variations and/or generalizations do not depart from the scope of the claimed subject matter.

Another feature of the systems and methods described herein is a post-processing of the data whereby the distribution of the posterior mean (or median) is approximated by a probability mixture model. In one embodiment involving classification over only two groups, the mixture may be, for example, a two-point probability density mixture. Alternatively, it may be, for example, that the two densities are normal with the same mean but differing variances, corresponding to the two different hypotheses. Using collected data, a fit is made to the theoretical mixture probability, and its parameters are determined.

The systems and methods described herein may be implemented on a computer system, with data on a computer-readable format such as a compact disk, a hard drive, a flash card, or a network server. Computer instructions can be written, either using commercially-available software packages (such as SAS) or using in-house custom-designed code, to implement the methods and systems described herein. Alternatively, in one embodiment, the data and/or the computer software that implements the methods and systems described herein may be hosted on a server, downloadable by an end user. In yet another embodiment, the data and the instructions may be downloadable from, and/or the software executable on, a distributed computing environment comprising one or more computers, network servers, or other data processing platforms. The various computerized configurations configured to implement the systems and methods described herein are readily apparent to those of ordinary skill in the art, after having read this specification.

Further features and advantages of the systems and methods described herein will be apparent from the following description of illustrative embodiments, and from the claims.

### BRIEF DESCRIPTION OF THE DRAWINGS

The following figures depict certain illustrative embodiments of the systems and methods described herein, in which like reference numerals refer to like elements. These depicted embodiments are to be understood as illustrative, and not as limiting in any way.

FIG. 1 depicts p-values from gene simulation model using p=10000 genes with a mean group separation of 1.5 and 0 for 10% and 90% of genes respectively.

FIG. 2a shows estimated values for |E(μⱼ|Y)| versus Var(μⱼ|Y) from the gene simulation model of FIG. 1.

FIG. 2b depicts absolute mean differences |\overline{Y}_{j,2}−\overline{Y}_{j,1}| versus Var(μⱼ|Y) for gene j.

FIG. 3a shows BAM test statistics μ_{j,n}* versus Z_{j,n} from simulations in FIG. 2.

FIG. 3b depicts a BAM test statistic by group plot.

FIG. 3c depicts a Z-test by group plot.

FIG. 4a shows the total number of misclassified observations from gene simulation model p=25000.

FIG. 4b shows a close-up view for smaller α values.

FIG. 5a depicts the total number of misclassified observations from the gene simulation model used in FIG. 4, but with smaller signal-to-noise ratio.

FIG. 5b plots the BAM test statistics against Z-tests similar to FIG. 3.

FIG. 6a shows gene mean values \overline{Y}_{j,l} versus gene sample variances \hat{σ}_{j,l}².

FIG. 6b shows group mean differences \overline{Y}_{j,2}−\overline{Y}_{j,1} versus standard errors.

FIG. 7a shows the total number of misclassified observations from the Poisson gene simulation model.

FIG. 7b shows the total number of misclassified observations from variance stabilized data.

FIG. 8a shows the gene mean values \overline{Y}_{j,l} versus gene standard deviations \hat{σ}_{j,l} from colon cancer data.

FIG. 8b shows the group mean differences \overline{Y}_{j,2}−\overline{Y}_{j,1} versus standard errors.

FIG. 8c shows genes found significant using Zcut with α=0.0005.

FIG. 9 depicts estimated values for |E(μⱼ|Y)| versus Var(μⱼ|Y) using weighted regression.

FIG. 10 shows genes found significant using Zcut with α=0.0005.

### DETAILED DESCRIPTION OF CERTAIN ILLUSTRATED EMBODIMENTS

To provide an overall understanding of the systems and methods described herein, certain illustrative practices and embodiments will now be described. However, it will be understood by one of ordinary skill in the art that the systems and methods described herein can be adapted and modified and applied in other applications, and that such other additions, modifications, and uses will not depart from the scope hereof.

Overview

Unless defined otherwise, all technical and scientific terms used herein have the same meaning as commonly understood by one of ordinary skill in the art to which the systems and methods described herein pertain.

The embodiments described herein comprise statistical data analysis methods, and the systems that implement those methods, which may be used in various contexts and applications. Although by no means limited to large-scale systems, in terms of scope, the systems and methods described herein are suitable for applications wherein a large body of data is collected, and there is a need to discern structure within the data for the purpose of classification, significance detection, or both. Accordingly, biomedical applications, such as gene array data analysis, wherein the problem of interest includes identifying which genes contribute to disease, for example, are natural contexts for classification and model selection.

Generally, prior art methods have had shortcomings in properly classifying data points that fall within some intermediate or “shady region” within the collected data set. This has been so at least in part because prior art methods have focused on keeping in check one type of classification error (false discovery or false alarm) at the expense of another classification error (false non-discovery).

The systems and methods described herein address these shortcomings, and improve upon the prior art by providing not only a theoretical (and hence objective) benchmark to classify data points that fall within some intermediate range for a particular context of interest, but also provide a graphical tool for a data analyst to use to develop rules of thumb, and to cultivate a better intuition for, the distribution of data, especially the intermediate-range data whose classification has caused trouble for prior art methods.

Employing a series of pre-processing, analysis, and post-processing methods, the systems and methods described herein not only address the concerns of prior approaches (in which false discovery rate was paramount in importance), but also disclose a method to control other sources of misclassification (a false non-discovery, for example).

To appreciate the scope of applicability of the systems and methods described herein, various contexts are described from which data can be obtained and to which the methods and systems described herein may be applied.

Generating Data for Analysis

Statistical methods described herein may be used to analyze a wide variety of data, including data generated by studies in the physical, biological or social sciences. While the statistical methods described herein are widely applicable, the methods will often provide particularly substantial advantages when applied to large data sets (e.g. data sets having greater that about 100, 1000 or 10,000 data points. Furthermore, methods disclosed herein may be particularly beneficial when there is reason to believe that it would be of interest to decrease the rate of false negative categorization in the data analysis.

In certain embodiments, statistical methods described herein may be applied to biomolecular expression data. The term “biomolecules” is used herein to refer to any of the various molecules produced by or present in a cell, a sample containing cells, a non-cellular or substantially non-cellular biological sample (e.g. a sample of extracellular material, such as hair) or organisms that have non-cellular states, such as viruses. “Expression data” is data that reflects the amount (e.g., relative or absolute, measured directly or indirectly) of one or more biomolecules present in a sample. Data sets that include expression data may also include other information, such as, for example, information about post-translational modifications on proteins. Biomolecules may be, for example, genomic DNA, RNAs (both mRNAs and other RNAs), lipids, carbohydrates, or other metabolites. Where the biomolecules to be studied are RNA transcripts, the data may be referred to as “gene expression data” or “DNA expression data”. Where the biomolecules to be studied are proteins, the data may be referred to as “protein expression data”.

A variety of methods for obtaining biomolecular expression data are well-known in the art. Several examples are described briefly here.

Gene Expression Data

In various aspects, the systems and methods described herein are suitable for analyzing gene expression data. Gene expression data may be gathered in any way that, in view of this specification, is available to one of skill in the art. Although many statistical methods provided herein are powerful tools for the analysis of data obtained by highly parallel data collection systems, methods disclosed herein are also useful for the analysis of data gathered by more traditional methods.

Many gene expression detection methodologies employ a polynucleotide probe which forms a stable hybrid with that of the target gene. If it is expected that the probes will be perfectly complementary to the target sequence, stringent conditions will often provide superior results. Lesser hybridization stringency may be used if some mismatching is expected, for example, if variants are expected with the result that the probe will not be completely complementary. Conditions may be chosen which rule out nonspecific/adventitious bindings, that is, which minimize noise.

Gene arrays are often used for highly parallel observation of many genes simultaneously. An array may comprise a set of spatially addressable probes that hybridize to several, and even thousands or more different transcripts. Such arrays are often classified as microarrays or macroarrays, and this classification depends on the size of each position on the array.

Probes in an array may be immobilized on or in a solid or semisolid support. Oligonucleotides can be bound to a support by a variety of processes, including lithography, and where the support is solid, it is common in the art to refer to such an array as a “chip”, although this parlance is not intended to indicate that the support is silicon or has any particular conductive properties. Nucleic acid probes may be spotted onto a substrate in a two-dimensional matrix or array, or a pre-fabricated array may be purchased from a manufacturer, such as Affymetrix (Santa Clara, Calif.). Samples of nucleic acids (e.g. RNAs or cDNAs derived from cells of interest) can be labeled and then hybridized to the probes. Nucleic acids, comprising the labeled or tagged sample nucleic acids bound to probe nucleic acids, can be detected once the unbound portion of the sample is washed away. In addition, a variety of corporations will perform microarray analysis on a contractual basis, such as Cellomics, Inc. (Pittsburgh, Pa.), GenHunter Corp. (Nashville, Tenn.) and Affymetrix.

In other embodiments, the sample nucleic acid is not labeled. In this case, hybridization can be determined, e.g., by plasmon resonance, as described, e.g., in Thiel et al. (1997) Anal. Chem. 69:4948.

When using commercially available microarrays, adequate hybridization conditions are provided by the manufacturer. When using non-commercial microarrays, adequate hybridization conditions can be determined based on the following hybridization guidelines, as well as on the hybridization conditions described in the numerous published articles on the use of microarrays.

Theory and practice of nucleic acid hybridization is described, e.g., in S. Agrawal (ed.) Methods in Molecular Biology, volume 20; and Tijssen (1993) Laboratory Techniques in biochemistry and molecular biology-hybridization with nucleic acid probes, e.g., part I chapter 2 “Overview of principles of hybridization and the strategy of nucleic acid probe assays,” Elsevier, New York provide a basic guide to nucleic acid hybridization.

Resultant hybridization patterns on an array may be visualized or detected in a variety of ways, with the particular manner of detection being chosen based on the particular label of the target nucleic acid, where representative detection means include scintillation counting, autoradiography, fluorescence measurement, calorimetric measurement, light emission measurement, light scattering, and the like.

One method of detection includes an array scanner that is commercially available from Affymetrix (Santa Clara, Calif.) or Agilent. Additional scanning devices are described in, for example, U.S. Pat. Nos. 5,143,854 and 5,424,186.

When fluorescently labeled probes are used, the fluorescence emissions at each site of a transcript array can be detected by scanning confocal laser microscopy. In one embodiment, a separate scan, using the appropriate excitation line, is carried out for each fluorophore used. Alternatively, a laser can be used that allows simultaneous specimen illumination at wavelengths specific to more than one fluorophore and emissions from more than one fluorophore can be analyzed simultaneously. Fluorescence laser scanning devices are described in Schena et al., 1996, Genome Res. 6:639-645 and in other references cited herein. Alternatively, the fiber-optic bundle described by Ferguson et al., 1996, Nature Biotech. 14:1681-1684, may be used to monitor mRNA abundance levels.

Following the data gathering operation, the data will typically be reported to a data analysis system. To facilitate data analysis, the data obtained by the reader from the device will typically be analyzed using a digital computer. Typically, the computer will be appropriately programmed for receipt and storage of the data from the device, as well as for analysis and reporting of the data gathered, e.g., subtraction of the background, deconvolution of multi-color images, flagging or removing artifacts, verifying that controls have performed properly, normalizing the signals, interpreting fluorescence data to determine the amount of hybridized target, normalization of background and single base mismatch hybridizations, and the like. Various analysis methods that may be employed in such a data analysis system, or by a separate computer are described herein.

While the above discussion focuses on the use of arrays for the collection of gene expression data, such data may also be obtained through a variety of other methods, that, in view of this specification, are known to one of skill in the art. The following are examples (not intended to be comprehensive) of alternative methods for gathering gene expression data.

A method for high throughput analysis of gene expression is the serial analysis of gene expression (SAGE) technique, first described in Velculescu et al. (1995) Science 270, 484-487. Among the advantages of SAGE is that it has the potential to provide detection of all genes expressed in a given cell type, whether previously identified as genes or not, provides quantitative information about the relative expression of such genes, permits ready comparison of gene expression of genes in two cells, and yields sequence information that can be used to identify the detected genes. Thus far, SAGE methodology has proved itself reliable in detecting expression of regulated and nonregulated genes in a variety of cell types (Velculescu et al. (1997) Cell 88, 243-251; Zhang et al. (1997) Science 276, 1268-1272 and Velculescu et al. (1999) Nat. Genet. 23, 387-388.

Gene expression data may be gathered by RT-PCR. mRNA obtained from a sample is reverse transcribed into a first cDNA strand and subjected to polymerase chain reaction (PCR). House keeping genes, or other genes whose expression is fairly constant can be used as internal controls and controls across experiments. Following the PCR reaction, the amplified products can be separated by electrophoresis and detected. Taqman™ fluorescent probes, or other detectable probes that become detectable in the presence of amplified product may also be used to quantitate PCR products. By using quantitative PCR, the level of amplified product will correlate with the level of RNA that was present in the sample. The amplified samples can also be separated on an agarose or polyacrylamide gel, transferred onto a filter, and the filter hybridized with a probe specific for the gene of interest. Numerous samples can be analyzed simultaneously by conducting parallel PCR amplification, e.g., by multiplex PCR.

Transcript levels may also be determined by dotblot analysis and related methods (see, e.g., G. A. Beltz et al., in Methods in Enzymology, Vol. 100, Part B, R. Wu, L. Grossmam, K. Moldave, Eds., Academic Press, New York, Chapter 19, pp. 266-308,1985). In one embodiment, a specified amount of RNA extracted from cells is blotted (i.e., non-covalently bound) onto a filter, and the filter is hybridized with a probe of the gene of interest. Numerous RNA samples can be analyzed simultaneously, since a blot can comprise multiple spots of RNA. Hybridization is detected using a method that depends on the type of label of the probe. In another dotblot method, one or more probes of one or more genes characteristic of disease D are attached to a membrane, and the membrane is incubated with labeled nucleic acids obtained from and optionally derived from RNA of a cell or tissue of a subject. Such a dotblot is essentially an array comprising fewer probes than a microarray.

Techniques for producing and probing nucleic acids are further described, for example, in Sambrook et al., “Molecular Cloning: A Laboratory Manual” (New York, Cold Spring Harbor Laboratory, 1989).

Protein Expression Data

In various aspects, the systems and methods described herein are suitable for analyzing protein expression data. Protein expression data may be gathered in any way that, in view of this specification, is available to one of skill in the art. Although many analytical methods provided herein are powerful tools for the analysis of protein data obtained by highly parallel data collection systems, many such methods are also useful for the analysis of data gathered by more traditional methods.

Immunoassays are commonly used to quantitate the levels of proteins in samples, and many other immunoassay techniques are known in the art. The invention is not limited to a particular assay procedure, and therefore is intended to include both homogeneous and heterogeneous procedures. Exemplary immunoassays which can be conducted according to the invention include fluorescence polarization immunoassay (FPIA), fluorescence immunoassay (FIA), enzyme immunoassay (EIA), nephelometric inhibition immunoassay (NIA), enzyme linked immunosorbent assay (ELISA), and radioimmunoassay (RIA). An indicator moiety, or label group, can be attached to the subject antibodies and is selected so as to meet the needs of various uses of the method which are often dictated by the availability of assay equipment and compatible immunoassay procedures. General techniques to be used in performing the various immunoassays noted above are known to those of ordinary skill in the art.

Protein levels may be detected by a variety of gel-based methods. For example, proteins may be resolved by gel electrophoresis, preferably two-dimensional electrophoresis comprising a first dimension based on pI and a second dimension of denaturing PAGE. Proteins resolved by electrophoresis may be labeled beforehand by metabolic labeling, such as with radioactive sulfur, carbon, nitrogen and/or hydrogen labels. If phosphorylation levels are of interest, proteins may be metabolically labeled with a phosphorus isotope. Radioactively labeled proteins may be detected by autoradiography, or by use of a commercially available system such as the PhosphorImager™ available from Molecular Dynamics (Amersham). Proteins may also be detected with a variety of stains, including but not limited to, Coomassie Blue, Ponceau S, silver staining, amido black, SYPRO dyes, etc. Proteins may also be excised from gels and subjected to mass spectroscopic analysis for identification. Gel electrophoresis may be preceded by a variety of fractionation steps to generate various subfractionated pools of proteins. Such fractionation steps may include, but are not limited to, ammonium sulfate precipitation, ion exchange chromatography, reverse phase chromatography, hydrophobic interaction chromatography, hydroxylapatite chromatography and any of a variety of affinity chromatography methods.

Protein expression levels may also be measured through the use of a protein array. For example, one type of protein array comprises an array of antibodies of known specificity to particular proteins. Antibodies may be affixed to a support by, for example, the natural interaction of antibodies with supports such as PVDF and nitrocellulose, or, as another example, by interaction with a support that is covalently associated with protein A (see for example U.S. Pat. No. 6,197,599), which binds tightly to the constant region of IgG antibodies. Antibodies may be spotted onto supports using technology similar to that described above for spotting nucleic acid probes onto supports. In another example, an array is prepared by coating a surface with a self-assembling monolayer that generates a matrix of positions where protein capture agents can be bound, and protein capture agents range from antibodies (and variants thereof) to aptamers, phage coat proteins, combinatorially derived RNAs, etc. (U.S. Pat. No. 6,329,209). Proteins bound to such arrays may be detected by a variety of methods known in the art. For example, proteins may be metabolically labeled in the sample with, for example, a radioactive label. Detection may then be accomplished using devices as described above. Proteins may also be labeled after being isolated from the sample, with, for example, a cross-linkable fluorescent agent. In one example, proteins are desorbed from the array by laser and subjected to mass spectroscopy for identification (U.S. Pat. No. 6,225,047). In another variation, the array may be designed for detection by surface plasmon resonance. In this case, binding is detected b changes in the surface plasmon resonance of the support (see, for example, Brockman and Fernandez, American Laboratory (June, 2001) p.37).

Metabolite Expression Data

In various aspects, the systems and methods described herein are suitable for analyzing metabolite expression data. Metabolites, such as lipids, carbohydrates, cofactors, amino acids and other types of molecules found in organism may be measured in a number of different ways, and such methods tend to vary depending on the type of compound to be analyzed. High throughput systems for metabolite analysis include liquid chromatography (e.g. reverse phase HPLC) coupled to a mass spectrophotometer and gas chromatography coupled to a mass spectrophotometer. Other traditional methods for analyzing metabolites may be employed, such as enzymatic assays, selective extractions and others known in the art.

Methods disclosed herein may also be used to analyze biomolecular data that may not typically be characterized as expression data. Biomolecular arrays are available, for example, from Santa Clara, Calif.-based Affymetrix. A survey of biomolecular arrays appears in “The Array of Today: Biomolecule arrays become the 21st century's test tube,” by J. D. Cortese, The Scientist, 14[17]:25, Sep. 4, 2000.

In one embodiment, biomolecular arrays are ordered molecular arrays that simplify discrimination of surface-bound biomolecules through the spatial control of ligand presentation. According to one practice, first, photolithography is used to spatially direct the synthesis of a matrix of biological ligands. A high-affinity binding partner is then applied to the matrix, which binds at locations defined by the ligand array. An atomic force microscope (AFM) may then be used to detect the presence and organization of the high-affinity binding partner.

A number of high throughput systems for genotype analysis are now available. An array of single-base extension tags (SBE-TAGs) has been described for the highly parallel analysis of single nucleotide polymorphisms present in a human genotype. Hirschhorn et al., Proc Natl Acad Sci USA. 2000 97(22):12164-69. SBE-TAGS may also be used for the analysis of other organisms. Similar arrays have been used to identify pathogens (e.g. viruses, protozoans or bacteria), for example, by hybridizing samples of genomic or transcribed nucleic acids to an array containing probes corresponding to different genomic regions of a variety of related pathogenic and nonpathogenic organisms. Borucki et al. Vet Microbiol 2003 92(4):351-62. Alkhalidi et al. J AOAC Int 2002 85(4):906-10. Wilson et al. Mol Cell Probes 2002 April;16(2):119-27.

Tissue Data

In various aspects, the systems and methods described herein are suitable for analyzing tissue array data, whereby multiple tissue cores are affixed, typically in the form of an array, to a chip, in a manner not unlike how genes are affixed to a gene array; a typical size of a tissue array is about 300 to about 400 cores, although other array sizes may be used in various alternative embodiments, without departing from the scope of the systems and methods disclosed herein.

Tissue arrays facilitate rapid evaluation, typically in parallel and in situ, of the role of normal and abnormal genes or gene products from hundreds of tissue samples. For example, studies involving tumors at various stages and disease grades improve the evaluation of the diagnostic, prognostic, and therapeutic characteristics of cancer gene candidates. In the absence of tissue arrays, such evaluation is frequently difficult, because it is typically conducted serially. Tissue microarrays provide a high-throughput means for the study of, for example, and without limitation, gene dosage and protein expression patterns in a large number of individual tissues for rapid and comprehensive molecular profiling of cancer and other diseases, without usurping limited tissue supplies.

In one practice, a tissue array application includes searching for oncogene amplifications in tumor tissue panels. Large-scale studies involving tumors encompassing differing stages and grades of disease facilitate a more efficient validation of putative markers that may ultimately correlate genotypes with phenotypes.

In one exemplary embodiment, hundreds of substantially cylindrical formalin fixed and paraffin-embedded tissue cores are densely and precisely arrayed into a typical paraffin block. From this, up to a few hundred serial sections may be produced and placed on individual glass microscopic slides. An adhesive-coated tape system (e.g., produced by Instrumedics, Hackensack, N.J.) may be useful in maintaining array formatting in the transfer of cut ribbons to the glass slides. Moreover, the tissue array technology is applicable to other medical research disciplines in which, for example, paraffin-embedded tissues are used, including, but not limited to, structural, developmental, and metabolic studies.

In general, by employing tissue arrays, various observations may be made about the tissues; for example, immunohistochemistry (IHC), morphology and protein expression along a particular pathway of interest. In particular, tests that may be performed using tissue arrays may include morphologic brightfield examination (H&E), protein expression studies (immunohistochemistry), DNA copy number analysis (fluorescence in situ hybridization FISH), or a combination thereof. Also, in situ PCR and traditional histologic special stains may be applied to tissue array slides. In some cases, mRNA level expression studies may also be performed.

The studies listed above may be done separately, or, more typically and effectively, substantially in parallel (for example, to concurrently compare gene amplification and expression in the same tissue). The systems and methods described herein are suitable for performing statistical analyses of data obtained from tissue arrays.

Viral Data

In various aspects, the systems and methods disclosed herein are suitable for analyzing viral data, whereby, for example, genomic sequences from a viral bank of viral genomes are printed onto an array, in a manner not unlike how genes are affixed to a gene array. These genomic sequences typically represent highly conserved sequences of viral DNA, which are characteristic of, or substantially specific to, a particular virus. This aspect is not necessarily associated with viral gene expression per se.

According to one practice, an unknown viral sample (e.g., from nasal lavage or sputum) is hybridized to the virus chip, and the pattern of hybridization is observed. Within the context of the systems and methods described herein, this analysis includes a one-way ANOVA. Of interest here is, among other things, which hybridizations are significantly different from zero; typically, this is not a 2-way or M-way ANOVA. Studies of viral data by using the systems and methods described herein have potential applications in bio-terrorism applications.

An exemplary embodiment, in the context of differential gene expression data, is now described as a two-way model selection problem. This is for illustrative purposes only, and should not be construed as limiting the scope of the invention in any way. Those of skill in the art will appreciate that the methods and systems described herein readily apply to other data analysis contexts, including multi-way classification problems.

DNA microarrays open up a broad new horizon for investigators interested in studying the genetic determinants of disease. The high throughput nature of these arrays, where differential expression for thousands of genes can be measured simultaneously, creates an enormous wealth of information, but also poses a challenge for data analysis because of the large multiple testing problem involved. The solution has generally been to focus on optimizing false-discovery rates while sacrificing power. The drawback of this approach is that more subtle expression differences will be missed, which might give investigators more insight into the genetic environment necessary for a disease process to take hold.

Disclosed herein is a method for detecting differentially expressed genes based on a high-dimensional model selection technique, Bayesian ANOVA for Microarrays (BAM), which strikes a balance between false rejections and false nonrejections. The approach is based at least in part on a weighted average of generalized ridge regression estimates that provides the benefits of using shrinkage estimation combined with model averaging. A graphical tool based on the amount of shrinkage is developed to visualize the trade-off between low false-discovery rates and finding more genes. Simulations are used to illustrate BAMs performance, and the method is applied to a large database of colon cancer gene expression data. A working hypothesis in the colon cancer analysis is that large differential expressions may not be the only ones contributing to metastasis; in fact, moderate changes in expression of genes may be involved in modifying the genetic environment to a sufficient extent for metastasis to occur. A functional biological analysis of gene effects found by BAM, but not by other false-discovery-based approaches, supports the hypothesis.

1. Introduction. DNA microarray technology allows researchers to measure the expression levels of thousands of genes simultaneously over different time points, different experimental conditions, or different tissue samples. It is the relevant abundance of the genetic product that provides surrogate information about the relative abundance of the cells' proteins. The differences in protein abundance are what characterize genetic differences between different samples. In the preparation of a DNA microarray sample, DNA or RNA molecules labeled with fluorescent dye are hybridized with a library of complementary strands fixed on a solid surface.

Generally, there are two major branches of chip technologies. Oligonucleotide arrays contain gene-specific sequences, called probes, about 20 bases long for each gene. The resulting fluorescence intensity from the hybridization process gives information about the abundance of the corresponding sample mRNA (a precursor to the cells proteins). The other type of array involves complementary DNA (cDNA), which can be spotted on nylon filters or glass slides. Complex mRNA probes are reverse-transcribed to cDNA at two sites for each gene and labeled with red control or green test fluorescent dyes. The ratio of red/green intensities represents the amount of RNA hybridizing at each site.

Although many analysis questions may be of interest, a commonly posed question asks for the detection of differentially expressing genes between experimental states, for example, between control samples and treatment samples, or between normal tissue samples and diseased tissue samples. Current approaches involve Bayes and empirical Bayes mixture analysis, and multiple hypothesis testing approaches with corrections designed to control the expected false discovery rate (FDR). The FDR is defined as the false-positive rate among all rejected (null) hypotheses; that is, the total number of rejected hypotheses where the null is in fact true, divided by the total number of rejected hypotheses. Researchers have provided a sequential p-value method to control the expected FDR. (Note: what others sometimes call the FDR is referred to herein as the expected FDR, a slight departure in terminology.) Given a set of independent hypothesis tests and corresponding p values, the method provides an estimate k, such that if one rejects those hypotheses corresponding to P₍₁₎, P₍₂₎, . . . , P₍ₖ₎, the k-ordered observed p values, then the FDR is, on average, controlled under some pre-chosen level. For convenience, this is referred to herein as the BH method.

ANOVA-based models are another way to approach the problem. The first ANOVA methods were developed to account for ancillary sources of variation when making estimates of relative expression for genes. More recently, an efficient approach casting the differential detection problem as an ANOVA model and testing individual model effects with FDR corrections has been developed. With all of these FDR applications, the methods work well by ensuring that an upper bound is met; however, a side effect is often a high false nondiscovery rate (FNR). The FNR is defined as the proportion of nonrejected (null) hypotheses that are incorrect. Researchers have shown that the BH method cannot simultaneously optimize the expected FDR and the expected FNR, implying that the method has low power. (The simulations in Sec. 6 of this disclosure also confirm this behavior.) An oracle threshold value was described by researchers, which improves on BH in the case where p-value distributions are independent two-point mixtures. It has been recognized that power for the BH approach could be improved. Typically, however, there is a price to be paid in terms of increased computation and some subjectiveness. Moreover, the largest relative power gains observed by researchers is typically realized when large proportions of genes are truly differentially expressed, a property that might not hold in some disease problems, because the number of genes expressed compared with dimension are expected to be small.

1.1. Larger Models Versus False Discovery.

The systems and methods described herein and employed for detecting gene expression changes use a Bayesian ANOVA for microarrays (BAM) technique that provides shrinkage estimates of gene effects derived from a form of generalized ridge regression and are suitable for high-dimensional model selection in linear regression problems. A key feature of the BAM systems and methods is that its output permits different model estimators to be defined, and each can be tailored to suit the various needs of the user. For example, for analysts concerned with false discovery rates, the systems and methods described herein construct an estimator that focuses on the FDR.

Also disclosed herein is a graphical method, based on the amount of shrinkage, which can be used to visualize the trade-off between a low FDR and finding more genes. This device can be used to select α cutoff values for model estimators. Selecting an appropriate α value is critical to the performance of any method used to detect differentially expressing genes. Simply relying on using preset α values, especially conventional values used in traditional problems, can be a poor strategy as such values may sometimes be magnitudes off from optimal ones. The case study example of Section 7 illustrates how small a may be in practice.

As a consequence of shrinkage and model averaging, the BAM method will be shown to strike a good balance between identifying large numbers of genes and controlling the number of falsely identified genes. This kind of property may be of great importance in the search for a colon cancer metastatic signature, a topic that will be touched upon more in Section 7 as one illustration of applicability of the systems and methods described herein.

Very little is currently known about the genetic determinants of colon cancer metastasis, although it is generally agreed that a genetic signature is complex. In fitting in with this general philosophy, the hypothesis adopted herein is that genes with large differential expressions may not be the only ones contributing to metastasis—that in fact, more moderate changes in expression of some genes might be sufficient to trigger the process.

Proving this hypothesis directly is difficult. A more reasonable surrogate hypothesis is that the genes showing more moderate changes in expression provide a suitable milieu for other metastatic events—accompanied by other genes showing much larger expression differences. This general principle may be at play in many diseases other than colon cancer; therefore, increased power in detecting differentially expressed genes becomes more important, and the systems and methods described herein address this need.

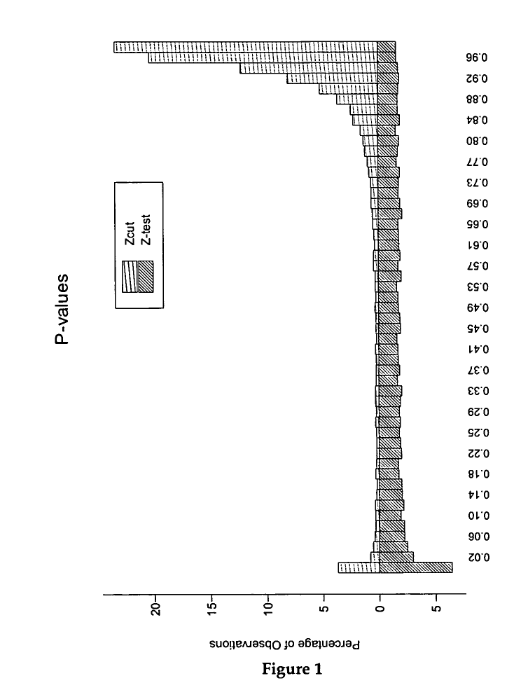

To fix ideas about BAM, consider Table 1 and FIG. 1 which present results from a gene simulation model involving p=10000 genes (specific details are provided in Section 6). FIG. 1 plots the histogram of the p-values from “Zcut” method (Section 3.3) against the p-values derived from individual Z-tests which use a pooled estimate for the standard error (Section 5.1). The figure shows the effect shrinkage plays in BAM; here it has the effect of pushing the p-value distribution for Zcut apart, helping to more clearly delineate expressive genes. Each gene had 5 observations per group. The top portion depicts p-values derived from the Zcut estimator, and the bottom portion includes data from standard Z-tests.

Differences in procedures can be carefully compared by Table 1, which records the number of genes detected as expressing, the FDR and FNR, and the Type-I and Type-II error rates. The Type-I error rate is the proportion of genes rejected given they are non-expressing (the null), while the Type-II error rate is the proportion of non-rejected genes given they are expressing (the alternative). Values are tabulated at an α=0.05 value. It should be noted how Zcut leads to a reduced FDR, while at the same time seeking to maintain high power. Table 1 also records the results of the BH method applied to the p-values from the Z-tests. Also recorded are the results from the “FDRmix” procedure (Section 4), a hybrid BH procedure. Table 1 shows that both BH and FDRmix lead to a smaller number of identified genes than Zcut or Z-test. This is at least in part because both procedures attempt to control the FDR, which typically results in fewer genes being found significant. Here, the BH method has the lowest FDR, slightly smaller than its target α=0.05 value. Although FDRmix is far off from the a target value, it does what its supposed to, which is to reduce the FDR of Zcut while maintaining power.

Care should be exercised in directly comparing FDR and FNR values, or for that matter Type-I and Type-II error rates, for the different procedures at the same α value, since an α target value means something different for each procedure. Looking at the different rates individually will does not, by itself, explain how the procedures perform overall. Thus, to be able to compare procedures on a more equal basis, an overall measure of performance is defined herein, “TotalMiss,” which is also recorded in Table 1. This is the total number of false rejections and false non-rejections, i.e. the number of misclassified genes for a procedure, which can be thought of as a measure of total risk. In terms of the total risk, Table 1 shows that Zcut is the clear winner here. A more detailed study of how TotalMiss varies with α will be presented in Section 6. This kind of analysis may be important in assessing the robustness of a procedure. Since α values can vary considerably depending upon the data, procedures that are robust will be those that exhibit uniformly good risk behavior.

1.2. Organization of the Remainder of the Disclosure.

This disclosure is organized as follows. Section 2 presents an overview of a stochastic variable selection algorithm that involves shrinkage of ordinary least squares estimators. The BAM procedure is introduced in Section 3. There the Zcut estimator and a shrinkage plot, which can be used as a graphical device for setting a values, are introduced. Theorems 1 and 2 of Section 3.4 discuss the robustness and adaptiveness of the BAM method and provide explanations for the Zcut method. Section 4 introduces the FDRmix procedure. Section 5 discusses some more commonly known procedures. The performance of BAM is studied via simulations in Section 6 and compared in detail to the more standard methods of Section 5. In Section 7 the colon cancer problem is discussed and a detailed analysis of the data based on BAM is presented. Section 8 concludes with a discussion, including a description of how the systems and methods described herein may be extended to analyze more than two groups.

2. The P-SVS procedure. An approach employed by the systems and methods described herein includes recasting the problem of finding differentially expressing genes as a problem of determining which factors are significant in a Bayesian ANOVA model. This is referred to herein as the BAM method. As one can generally rewrite an ANOVA model as a linear regression model, the task of finding expressive genes can be conceptualized as a variable selection problem. This connection is employed to advantage by the systems and methods described herein to select variables in high-dimensional regression problems called P-SVS (Parametric Stochastic Variable Selection). Section 3 outlines the details of the BAM approach and how it relates to P-SVS. A general description of the P-SVS procedure is now presented.

The P-SVS method is a hybrid version of the spike and slab models. It includes a Bayesian hierarchical approach for model selection in linear regression models,
Yᵢ=xᵢ^{T}β₀+εᵢ, i=1, . . . , n, (1)
where Yᵢ is the response value, εᵢ are i.i.d N(0, σ₀²) measurement errors and β₀=(β_{1,0}, . . . , β_{p,0})^{T} is the unknown (true) p-dimensional vector of coefficients for the covariates xᵢ. To answer the question of which β_{j,0} are nonzero, the P-SVS approach works by modeling (1) as a hierarchical model

$$\begin{matrix}( {Y}_{i} \vert β , {σ}^{2} , {x}_{i} ) {\sim}^{ind} Normal ( {x}_{i}^{T} β , {σ}^{2} ) , i = 1 , … , n \\ ( {β}_{j} \vert {γ}_{j} , {τ}_{j}^{2} ) {\sim}^{ind} Normal ( 0 , {γ}_{j} {τ}_{j}^{2} ) , j = 1 , … , p \\ ( {γ}_{j} \vert {γ}^{*} , λ ) {\sim}^{iid} ( 1 - λ ) {δ}_{{γ}^{*}} ( \cdot ) + {λδ}_{1} ( \cdot ) \\ ( {τ}_{j}^{- 2} \vert {b}_{1} , {b}_{2} ) {\sim}^{iid} Gamma ( {b}_{1} , {b}_{2} ) \\ λ \sim Uniform [ 0 , 1 ] \\ ( {σ}^{- 2} \vert {a}_{1} , {a}_{2} ) \sim Gamma ( {a}_{1} , {a}_{2} ) .\end{matrix} \qquad ( 2 )$$

A key feature of the model is the choice for the priors of τⱼ² and γⱼ which are calibrated so that the variance νⱼ²=γⱼτⱼ² for a coefficient βⱼ has a bimodal distribution. A large value for νⱼ² occurs when γⱼ=1 and τⱼ² is large and will induce large values for βⱼ, thus identifying the covariate as potentially informative. Small νⱼ² occur when γⱼ=γ* (chosen to be a small value). In this case the value for βⱼ will become near zero, signifying that βⱼ is unlikely to be informative. The value for λ in (2) controls how likely it is for γⱼ to be 1 or γ*, and thus it controls how many βⱼ are non-zero, and so it controls the complexity of the model. Generally, the choice of hyperparameters for priors is important to the behavior of νⱼ², and hence P-SVS's ability to properly select variables. The values for hyperparameters γ*, a₁, a₂, b₁, b₂ need not be tuned for each data set, and may be kept fixed.

Let γ=(γ₁, . . . , γₚ)^{T}. This is a way of encoding models in binary strings (if γⱼ=1 select βⱼ). One approach used in spike and slab variable selection looks to the posterior behavior of γ to identify the “best model”, for example by identifying the γ which occurs with highest posterior probability. However, in very large variable selection problems information is best processed differently as the information contained in γ is too coarse (if p is very large, a high frequency model may not even be found). Variables are selected by considering the magnitude of their posterior mean values. Motivation to use the posterior mean to select variables stems from its interpretation as an adaptive weighted average of generalized ridge estimates. Such values are reliable as they are produced by model averaging in combination with shrinkage, two methods that improve model selection. It can easily be seen that the posterior mean is a weighted ridge estimate. Let β=(β₁, . . . , βₚ)^{T}. From (2), the posterior mean can be written as
E*β|Y)=∫∫βπ(dβ|ν²,σ²,Y)π(dγ,dτ²,dλ,dσ²|Y),
where ν²=(ν²₁, . . . ,νₚ²)^{T}, τ²=(τ²₁, . . . ,τₚ²)^{T} and Y=(Y₁, . . . ,Yₙ). Elementary calculations show that
(β|ν²,σ²,Y)˜Normal(Σ⁻¹X^{T}Y,σ²Σ⁻¹), (3)
where X is the n×p design matrix, Σ=σ²Γ⁻¹+X^{T}X and Γ is the p×p diagonal matrix with diagonal values νⱼ²=γⱼτⱼ². Notice that the conditional posterior mean for β is
E(β|ν²,σ²,Y)=Σ⁻¹X^{T}Y=(σ²Γ⁻¹+X^{T}X)⁻¹X^{T}Y,
which is the the ridge estimate from a generalized ridge regression of Y on X with weights σ²Γ⁻¹. Small values for diagonal elements of Γ have the effect of shrinking coefficients. Thus, the posterior mean for β can now seen to be a weighted average of ridge shrunken estimates where the adaptive weights are determined from the posteriors of γ, τ² and λ.

This shift from high frequency models selected by γ to models selected on the basis of individual performance of variables is corroborated by other researchers. It has been shown that the high frequency model can be suboptimal even in the simplest case when the design matrix is orthogonal. Under orthogonality, the optimal predictive model is not the high frequency model, but the median probability model defined as the model consisting of variables whose overall posterior probability is greater than or equal to ½.

3. Bayesian ANOVA for microarrays. The BAM systems and methods described herein apply the variable selection device by recasting the microarray data problem in terms of ANOVA, and hence as a linear regression model. Consider the case wherein l=2 is the number of groups of samples; extensions to allow for more groups are discussed later in section 8. For group l, let Y_{j,k,l} denote the k-th expression value, k=1, . . . , n_{j,l}, for gene j=1, . . . , p. Group l=1 will correspond to the control group, while l=2 represents the treatment group. For example, in the colon cancer study of Section 7, the control group represents Duke B-survivor colon tissue samples while the treatment group are metastasized colon cancer samples. Microarray data, as in our colon cancer study, will often be collected from balanced experimental designs with fixed group sizes n_{j,1}=N₁ and n_{j,2}=N₂. In such settings, Y_{1,k,l}, . . . , Y_{p,k,l} will typically represent the p gene expressions obtained from a microarray chip for a specific individual k with a tissue type from group l (i.e. either an individual k from the control group or an individual k from the treatment group). However, since more complex experimental designs are possible the problem is approached herein more generally by allowing for unbalanced data. A key question of general interest is which genes are expressing differently over the two groups. Let ε_{j,k,l} be i.i.d N(0, σ₀²) measurement errors. The problem may be formulated as the ANOVA model,
Y_{j,k,l}=θ_{j,0}+μ_{j,0}I{l=2}+ε_{j,k,l}, j=1, . . . ,p, k=1, . . . ,n_{j,l}, l=1,2 (4)
where those genes that are expressing differentially correspond to indices j where μ_{j,0}≠0 (if genes turn on then μ_{j,0}>0, otherwise if they turn off μ_{j,0}<0).

Observe that (4) has 2p parameters. Many of the θ_{j,0} parameters will be non-zero since they estimate the overall mean for a gene j. However, since our interest is only in μ_{j,0}, the gene-treatment effect, it is convenient to force θ_{j,0} to be near zero so that the effective dimension of the problem is p. A useful strategy is to replace Y_{j,k,l} by its centered value
Y_{j,k,l}−\overline{Y}_{j,1}, (5)
where

$${\overline{Y}}_{j , l} = {\sum }_{k = 1}^{{n}_{j , l}} {Y}_{j , k , l} / {n}_{j , l}$$

is the mean for gene j over group l. This will also reduce the correlation between the Bayesian parameters θⱼ and μⱼ and greatly improves the calibration of P-SVS. Section 3.2 will explain this point in more detail.

Extensions to (4) are possible. For example, additional covariates may be included in the model, which is useful in experiments where external data (such as clinical information of individuals providing tissue samples) is collected alongside gene expressions. Thus (4) may be extended to the important class of ANCOVA (analysis of covariance) models. In another extension, genes may have different variability in addition to different mean expression levels. In such cases, (4) may be extended to allow for differing gene measurement error variances σⱼ². Often σⱼ² is related to the mean gene expression. In settings where this relationship is simple, for example, where the variance might be linear in the mean, this behavior is often handled by applying a variance stabilizing transform. Section 6.1 will study how well this approach works. On, the other hand, the relationship between σⱼ² and the mean expression may be complex, as is the case for the colon cancer study of Section 7. For such problems a specialized method will be developed.

The expressions Y_{j,k,l} are typically the end product of some form of probe-level standardization across samples (i.e. chips). Standardization for the colon cancer data involved standardizing chips to a gamma distribution with a fixed shape parameter. Further gene-level pre-processing is done as discussed below. With so much processing of the data it is natural to question the assumption of normality in (4). In fact, Theorem 1 of Section 3.4 will show that this assumption is not necessary due to the effect of a central limit theorem, as long as ε_{j,k,l} are independent with suitably behaved moments and if group sizes n_{j,l} are relatively large. Some empirical evidence of this effect will be presented in Section 6 for Poisson data.

3.1. Linear regression and preprocessing the data. To harness P-SVS in the BAM method, (4) is rewritten as a linear regression model (1), that is, using a single index i. This is accomplished by stringing observations (5) out in order, starting with the observations for gene j=1, group l=1, followed by values for gene j=1, group l=2, followed by observations for gene j=2, group l=1, etc. Notationally, the following parameter adjustments are also made. In place of βⱼ, coefficients (θⱼ, μⱼ) are used. Hierarchical prior variances for (θⱼ, μⱼ) are now denoted by (ν₂ⱼ₋₁², ν₂ⱼ²). Analogous notational changes are applied to γ and τ² in (2).

The second step to using P-SVS requires a data pre-processing step which rescales the observations by the square-root of the sample size divided by the mean square error and by transforming the design matrix so that its columns each satisfy Σᵢx_{i,j}²=n. This calibrates the conditional variance (Γ⁻¹+X′X/σ²)⁻¹ for β in (3) to have diagonal elements nearly zero or one in low to moderate correlation problems. Variances of zero correspond to non-significant variables, while variances of one correspond to informative covariates. Since the conditional variance of one represents a good lower bound for significant variables, one can then use a standard N(0, 1) distribution to assess whether a specific regression coefficient βⱼ should be considered informative, and hence included in the model.

Let \hat{σ}ₙ² denote the usual unbiased estimator for σ₀² from (4),

$${\hat{σ}}_{n}^{2} = \frac{1}{n - 2 p} {\sum }_{j , k , l}^{} {( {Y}_{j , k , l} - {\overline{Y}}_{j , 1} I \{ l = 1 \} - {\overline{Y}}_{j , 2} I \{ l = 2 \} )}^{2} ,$$

where

$$n = {\sum }_{j = 1}^{p} {n}_{j}$$

are the total number of observations and nⱼ=n_{j,1}+n_{j,2} is the total sample size for gene j. To calibrate the data, replace the observations (5) with rescaled values
\tilde{Y}_{j,k,l}=(Y_{j,k,l}−\overline{Y}_{j,1})×\sqrt{n/\hat{σ}}ₙ².

The effect of this scaling will be to force 2 to be approximately equal to n and it will rescale posterior mean values so they can be directly compared to a limiting normal distribution. Columns are also rescaled so they have second moments equal to one. Thus after pre-processing, (4) may be rewritten as
\tilde{Y}=\tilde{X}\tilde{β}₀+\tilde{ε},
where \tilde{Y} is the vector of the n strung out \tilde{Y}_{j,k,l} values, \tilde{β}₀ is the new regression coefficient under the scaling, \tilde{ε} is the new vector of measurement errors and \tilde{X} is the n×(2p) design matrix. Here \tilde{X} is defined so that its 2ⱼ-1 column consists of zeroes everywhere except for the values \sqrt{n/nⱼ} placed along the nⱼ rows corresponding to gene j, while column 2j consists of zeroes everywhere except for values \sqrt{n/n_{j,2}} placed along the n_{j,2} rows corresponding to gene j for group l=2.

3.2. Ridge regression estimates. Because of the simple nature of the design matrix \tilde{X} the conditional distribution for β may be explicitly determined. Adjusting to the new notation, a little bit of algebra using (3) shows that

$$\begin{matrix}( {( {θ}_{j} , {μ}_{j} )}^{T} \vert {v}_{2 j - 1}^{2} , {v}_{2 j}^{2} , {σ}^{2} Y ) \sim Normal ( {( {\hat{θ}}_{j , n} , {\hat{μ}}_{j , n} )}^{T} , {\hat{\sum }}_{j , n}^{- 1} ) , \\ where & \\ ( \begin{matrix}{\hat{θ}}_{j , n} \\ {\hat{μ}}_{j , n}\end{matrix} ) = \frac{n {\hat{\sum }}_{j , n}^{- 1}}{{\hat{σ}}_{n} {σ}^{2}} ( \begin{matrix}{n}_{j}^{- 1 / 2} {\sum }_{k , l} ( {Y}_{j , k , l} - {\overline{Y}}_{j , 1} ) \\ {n}_{j , 2}^{- 1 / 2} {\sum }_{k} ( {Y}_{j , k , 2} - {\overline{Y}}_{j , 1} )\end{matrix} ) , \\ and & ( 6 ) \\ {\hat{\sum }}_{j , n} = ( \begin{matrix}n / {σ}^{2} + 1 / {v}_{2 j - 1}^{2} & n / {σ}^{2} \times \sqrt{{n}_{j , 2} / {n}_{j}} \\ n / {σ}^{2} \times \sqrt{{n}_{j , 2} / {n}_{j}} & n / {σ}^{2} + 1 / {v}_{2 j}^{2}\end{matrix} ) . & \end{matrix}$$

Recall that as a consequence of the pre-processing step, σ² is approximately equal to n. Also, since \hat{θ}_{j,n} is nearly zero due to the centering (5), it is expected that υ₂ⱼ₋₁²≈0. Thus,

$${\hat{\sum }}_{j , n}^{- 1} \approx ( \begin{matrix}0 & 0 \\ 0 & {v}_{2 j}^{2} / ( {v}_{2 j}^{2} + 1 )\end{matrix} ) .$$

This implies that the posterior correlation between θⱼ and μⱼ are nearly zero. Thus, the centering method (5) has two important roles: (i) it reduces the dimension of the parameter space and (ii) it reduces correlation between parameters.

Now for genes that are expressing differently, it is expected that υ₂ⱼ² is large, and thus the conditional mean for μⱼ is approximately

$${\hat{u}}_{j , n} \approx \frac{\sqrt{{n}_{j , 2}}}{{\hat{σ}}_{n}} ( {\overline{Y}}_{j , 2} - {\overline{Y}}_{j , 1} ) \qquad ( 7 )$$

and the conditional variance for μⱼ is approximately υ₂ⱼ²/(υ₂ⱼ²+1)≈1. As the conditional variance represents a lower bound for the posterior variance V(μⱼ|Y) this suggests that the posterior mean E(μⱼ|Y) is to be compared to a N(0, 1) distribution to test whether μⱼ is essentially zero. However, Theorem 1 of Section 3.4 will show that it is more appropriate to use the theoretical limiting variance for \hat{μ}_{j,n} as our lower bound; here equal to nⱼ/n_{j,1}. Thus, E(μⱼ|Y) is to be compared to a N(0, nⱼ/n_{j,1}) distribution to test whether μ_{j,0} is non-zero. This is the basis of the Zcut procedure.

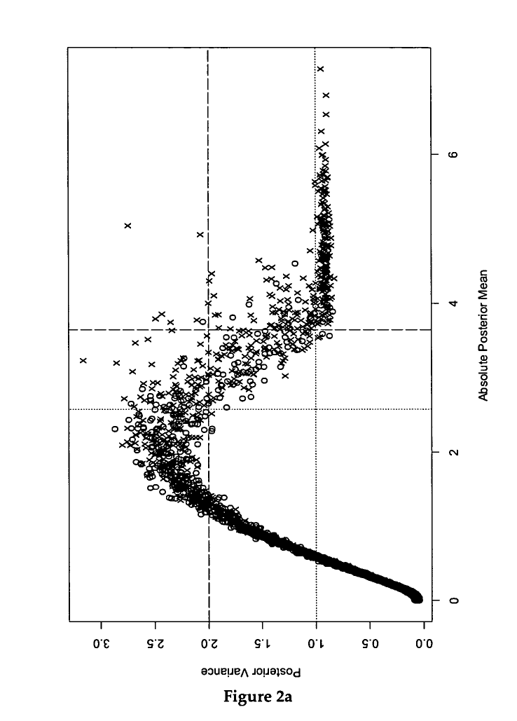

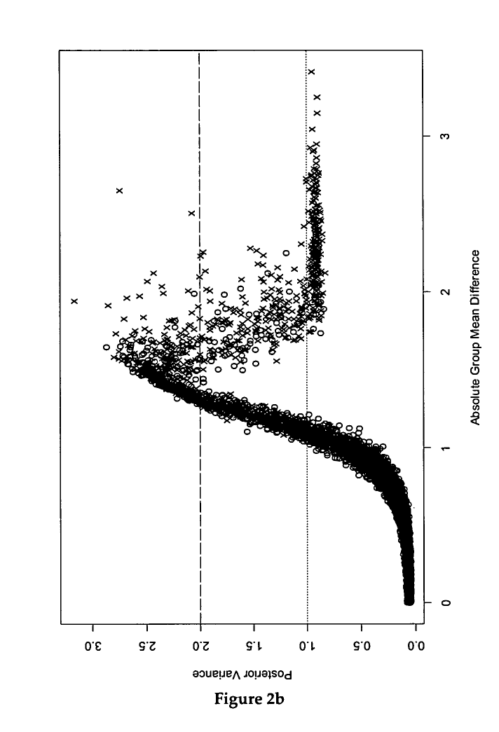

Theorem 1 justifies this strategy more rigorously. In the meantime, some evidence for this approach may be seen in FIG. 2(a) where the posterior absolute means and posterior variances from the earlier gene simulation model are plotted. In this “shrinkage plot” the lower variance bound of one is represented by a thin dashed horizontal line, while the thick dashed horizontal line represents the theoretical variance nⱼ/n_{j,1}=2. Circles indicate genes whose true means μ_{j,0}=0, while crosses are used for μ_{j,0}≠0. Notice how large values for |E(μⱼ|Y)| have posterior variances near one, which then jump up to the theoretical variance nⱼ/n_{j,1}=2 as |E(μⱼ|Y)| becomes moderate in size, finally dropping down to about zero as |E(μⱼ|Y)| becomes small.

FIG. 2(b) shows that the genes with large posterior variances are those with group mean differences that are intermediate in size. Thus, the jump is seen because the BAM systems and methods described herein shrink the posterior means while inflating the variance for these intermediate values, making it harder to reject the null μ_{j,0}=0. To better classify those genes with moderate values for |E(μⱼ|Y)| the variance is adjusted to equal nⱼ/n_{j,1}. As illustration, consider the two vertical lines in FIG. 2(a). The thin dashed line is the value for the 99.5 percentile from a standard N(0, 1) distribution, while the thick dashed line denotes the same percentile from a N(0, 2) distribution. Observe how the adjustment to the variance helps to avoid misclassifying genes.

It it is instructive to work out what the posterior conditional variance is without the use of the centering method (5). It is expected that \hat{θ}_{j,n} be nonzero and that υ₂ⱼ₋₁² be large. If μ_{j,0} is non-zero, then υ₂ⱼ² will be large, and hence

$${\hat{\sum }}_{j , n}^{- 1} \approx ( \begin{matrix}{n}_{j} / {n}_{j , 1} & - \sqrt{{n}_{j} {n}_{j , 2}} / {n}_{j , 1} \\ - \sqrt{{n}_{j} {n}_{j , 2}} / {n}_{j , 1} & {n}_{j} / {n}_{j , 1}\end{matrix} ) .$$

Note the resulting non-zero posterior correlation of −\sqrt{n_{j,2}/nⱼ} between θⱼ and μⱼ.

3.3. The Zcut procedure. Formally, is referred to herein as the Zcut procedure, or simply, Zcut, includes the following method for identifying parameters μ_{j,0} which are not zero (and hence genes j which are expressing). Identify a gene j as differentially expressing if
|E(μⱼ|Y)|≧z_{α/2}\sqrt{nⱼ/n_{j,1},}
where z_{α/2} is the 100×(1−α/2) percentile of a standard normal distribution. The value E(μⱼ|Y) is obtained by averaging Gibbs sampled draws for μⱼ.

In practice an appropriate way to select an α value for Zcut is employed. A technique used in Section 7 includes selecting a on the basis of a shrinkage plot, like FIG. 2(a). In Section 7, α is chosen to coincide with the vertical quantile so that most of the observations to its right will have posterior variance, Var(μⱼ|Y), nearly equal to one. This removes many intermediate values which could inflate the FDR.

3.4. Robustness and adaptiveness. The Zcut technique is justified by way of a central limit theorem. This analysis will justify the adjustment to the variance used in FIG. 2(a), identifying it with the asymptotic variance for the conditional posterior mean. These results hold even when measurement errors are not normally distributed, assuming that appropriate moment conditions are satisfied. In the following theorem note carefully the degeneracy of the limiting distribution. This is a consequence of the centering method (5).

THEOREM 1. Assume that (4) represents the true model where ε_{j,k,l} are independent random variables such that E(ε_{j,k,l})=0, E(ε_{j,k,l}²)=σ₀² and E(|ε_{j,k,l}³|), E(ε_{j,k,l}⁴)≦C₀ for some fixed constant C₀<∞. If σ²/n→1 and n_{j,1}→∞ and n_{j,2}→∞ for each j such that n_{j,2}/n_{j,1}→r_{j,0}<∞, then under the null hypothesis μ_{j,0}=0, keeping (υ₂ⱼ₋₁², υ₂ⱼ²) fixed,

$${( {\hat{θ}}_{j , n} , {\hat{μ}}_{j , n} )}^{T} {}_{} {Z}_{j} {\sum }_{j}^{- 1} {( \sqrt{{r}_{j , 0}} , \sqrt{1 + {r}_{j , 0}} )}^{T} , j = 1 , … , p , \\ where$$

$${\sum }_{j} = ( \begin{matrix}1 + 1 / {v}_{2 j - 1}^{2} & \sqrt{{r}_{j , 0} / ( 1 + {r}_{j , 0} )} \\ \sqrt{{r}_{j , 0} / ( 1 + {r}_{j , 0} )} & 1 + 1 / {v}_{2 j}^{2}\end{matrix} )$$

and Zⱼ are independent Normal(0, 1) random variables.

PROOF OF THEOREM 1. With a little bit of rearrangement in (6) it can be shown that

$${( {\hat{θ}}_{j , n} , {\hat{μ}}_{j , n} )}^{T} = {M}_{j , n} \sqrt{{n}_{j , 2}} ( {\overline{Y}}_{j , 2} - {\overline{Y}}_{j , 1} ) , where \\ {M}_{j , n} = \frac{n {\hat{\sum }}_{j , n}^{- 1}}{{\hat{σ}}_{n} {σ}^{2}} {( \sqrt{{n}_{j , 2} / {n}_{j} ,} 1 )}^{T} . \qquad ( 8 )$$

Because the third moment of ε_{j,k,l} is bounded a Liapounov Central Limit Theorem may be applied to each of the group averages \overline{Y}_{j,l}. Use the fact that the averages are independent, and that \sqrt{n_{j,2}/n_{j,1}}→\sqrt{τ_{j,0}}, to deduce that under the null, \sqrt{n_{j,2}}(\overline{Y}_{j,2}−\overline{Y}_{j,1}) [symbol] Normal(0,σ₀²(1+r_{j,0})). Meanwhile, some algebraic manipulation shows that

$${\hat{σ}}_{n}^{2} = \frac{1}{n - 2 p} {\sum }_{j , k , l}^{} {ε}_{j , k , l}^{2} - \frac{1}{n - 2 p} {\sum }_{j , l}^{} {n}_{j , l} {\overline{ε}}_{j , l}^{2} ,$$

where

$${\overline{ε}}_{j , l} = {\sum }_{k = 1}^{{n}_{j , l}} {ε}_{j , k , l} / {n}_{j , l} .$$

A bounded fourth moment implies that the first term on the right converges in probability to σₒ², while for the second term, a second moment ensures that \overline{ε}_{j,l}² [symbol] 0 for each j and l. Hence it follows that \hat{σ}ₙ² [symbol] σ₀². Therefore, since σ²/n→1, it follows that

$${M}_{j , n} {\to }^{p} {σ}_{0}^{- 1} {\sum }_{j}^{- 1} {( \sqrt{{r}_{j , 0} / ( 1 + {r}_{j , 0} )} , 1 )}^{T} .$$

Putting the pieces together and appealing to Slutsky's Theorem gives the desired result. Note that the degeneracy of the limit poses no problem by applying the Cramer Wold device.□

Most of the conditions of Theorem 1 are likely to hold in practice. Relaxing the assumption that errors are normally distributed and i.i.d is an especially helpful feature. The condition that σ²/n→1 is quite realistic and understandable due to the rescaling employed by the systems and methods described herein. It holds quite accurately in many problems. For example, in the simulations presented in FIG. 2(a), σ²/n had a posterior mean of 0.96 with a posterior standard deviation of 0.04. The value for r_{j,0} appearing in the theorem represents the limiting ratio of the group sizes. In the previous simulation r_{j,0}≈n_{j,2}/n_{j,1}=1. If gene j is really expressing, i.e. μ_{j,0}≠0, then it is expected that υ₂ⱼ² to be large, and hence Theorem 1 and an argument similar to (7) shows that \hat{μ}_{j,n} can be approximated in distribution by a Normal(0, 1+r_{j,0}), or roughly a Normal(0, 2), under the null. Notice how this matches up with the adjusted variance used in the simulation. In general, under the conditions of Theorem 1, \hat{μ}_{j,n}\sqrt{n_{j,1}/nⱼ} can be approximated by a standard normal distribution under the null when υ₂ⱼ² is large. Integrating over the hyperparameters yields μ_{j,n}*:=E(μⱼ|Y)\sqrt{n_{j,1}/nⱼ}, which can be thought of as a “Bayes test statistic.” Under the null it has a standard normal distribution. This is the rationale for the Zcut rule defined in Section 3.3. Theorem 1 shows that BAM is robust to underlying model assumptions and provides an explanation for the Zcut rule.

Theorem 2 below translates BAM's ability to adaptively shrink coefficients into a statement about risk performance. Shrinkage of coefficients implies shrinkage of test statistics. BAM's adaptiveness allows it to shrink the Bayes test statistics μ_{j,n}* for coefficients μ_{j,0}=0 while allowing μ_{j,n}* from coefficients μ_{j,0}≠0 to have values similar to frequentist Z-tests

$${Z}_{j , n} = \frac{{\overline{Y}}_{j , 2} - {\overline{Y}}_{j , 1}}{{\hat{σ}}_{n} \sqrt{1 / {n}_{j , 1} + 1 / {n}_{j , 2}}} . \qquad ( 9 )$$

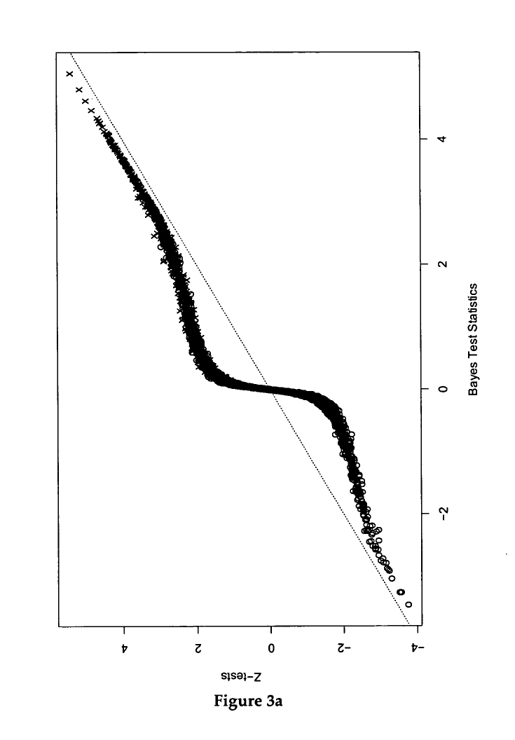

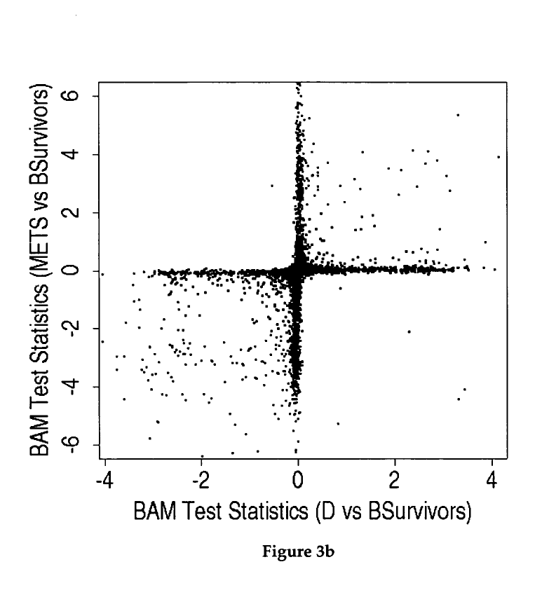

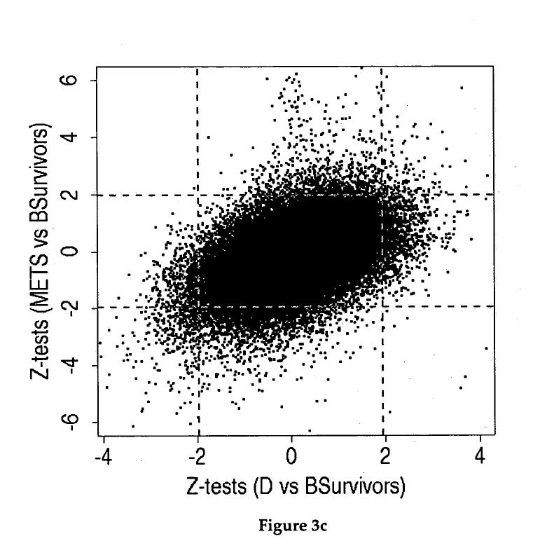

This has a direct impact on performance. Since both μ_{j,n}* and Z_{j,n} are compared to a Normal(0, 1) in assessing significance, Zcut will produce p-values that match up closely with Z-tests for non-zero coefficients, while p-values from the zero coefficients will be much larger. Recall that this p-value effect was seen in FIG. 1. FIG. 3a illustrates the effect in terms of test statistics. Expressed genes are indicated by a cross, while non-expressed genes are identified by circles (these are the values mostly near zero on the x-axis which have been shrunken by BAM). This translates into power for Zcut and a low FDR.

Theorem 2 below quantifies this adaptiveness in terms of risk behavior. By way of introducing some notation, let \hat{d}_{j,α} ε{0, 1} represent the classification rule for some two-sided test at a fixed α level. A value \hat{d}_{j,α}=1 means the null μ_{j,0}=0 is rejected, otherwise if \hat{d}_{j,α}=0 the null is accepted. Let
R_{j,α}=Pr{\hat{d}_{j,α}=1,μ_{j,0}=0}+Pr{\hat{d}_{j,α}=0,μ_{j,0}≠0}.

This is the expected risk for gene j. The total expected risk for all genes is R(α)=Σⱼ₌₁^{p} R_{j,α}. The average R(α)/p is the misclassification rate. Write R_{B}(α) and R_{F}(α) for the total expected risk using μ_{j,n}* and Z_{j,n} respectively for some fixed α value.

THEOREM 2. Assume that (4) represents the true model where ε_{j,k,l} are i.i.d Normal(0, σ₀²) variables. Suppose that σ²=n. Then for each 0<δ<½ there exists values (υ₂ⱼ₋₁², υ₂ⱼ²) for j=1, . . . ,p such that R_{B}(α)<R_{F}(α) for each α ε|δ,1−δ|.

PROOF OF THEOREM 2. Let I₀={j: μ_{j,0}=0} denote the indices for the zero coefficients. Define p₀ to be the cardinality of I₀. Since errors are assumed to be normally distributed, (9) implies that Z_{j,n} [symbol] ZⱼCₙ, where Cₙ=σ₀/\hat{σ}ₙ and Zⱼ are independent Normal(μ_{j,0}/S_{0,n}, 1) variables where S_{0,n}=σ₀\sqrt{1/n_{j,1}+1/n_{j,2}}. It follows that

$$\begin{matrix}{R}_{F} ( α ) = {\sum }_{j ∈ {I}_{0}}^{} Pr \{ \vert {Z}_{j , n} \vert \geq {z}_{α / 2} \} + {\sum }_{j ∈ {I}_{0}^{c}}^{} Pr \{ \vert {Z}_{j , n} \vert < {z}_{α / 2} \} \\ = {p}_{0} Pr \{ \vert N ( 0 , 1 ) \vert \geq {C}_{n}^{- 1} {z}_{α / 2} \} + {\sum }_{j ∈ {I}_{0}^{c}}^{} Pr \{ \vert {Z}_{j} \vert < {C}_{n}^{- 1} {z}_{α / 2} \} .\end{matrix}$$

Recall that (\hat{θ}_{j,n}, \hat{μ}_{j,n})^{T} can be written as (8). One can show that (remember σ²=n):
\hat{σ}ₙM_{j,n}→(0,υ₂ⱼ²/(1+υ₂ⱼ²))^{T}, as υ₂ⱼ₋₁²→0.

Because μ_{j,n}*=\hat{μ}_{j,n}\sqrt{n_{j,1}/nⱼ}, use (8) and the definition of Z_{j,n} to deduce that for each 0<ξⱼ<1 we can find a υ₂ⱼ₋₁², and υ₂ⱼ² such that μ_{j,n}*=Z_{j,n}ξⱼ. In particular, this means that for each 0<δ₁<1 and 0<δ₂<1, (υ₂ⱼ₋₁²,υ₂ⱼ²) may be found such that μ_{j,n}*=Z_{j,n}δ₁ for j ε I₀ and μ_{j,n}*=Z_{j,n}δ₂ for j ε I₀^{c}. Therefore,

$${R}_{F} ( α ) - {R}_{B} ( α ) = {p}_{0} ( Pr \{ \vert N ( 0 , 1 ) \vert \geq {C}_{n}^{- 1} {z}_{α / 2} \} - Pr \{ \vert N ( 0 , 1 ) \vert \geq {δ}_{1}^{- 1} {C}_{n}^{- 1} {z}_{α / 2} \} ) + {\sum }_{j ∈ {I}_{0}^{c}}^{} ( Pr \{ \vert {Z}_{j} \vert < {C}_{n}^{- 1} {z}_{α / 2} \} - Pr \{ \vert {Z}_{j} \vert < {δ}_{2}^{- 1} {C}_{n}^{- 1} {z}_{α / 2} \} ) .$$

Both sums on the right-hand side are continuous functions of α and each has a minimum and maximum over α ε[δ, 1−δ]. In particular, the second sum may be made arbitrarily close to zero uniformly over α ε[δ,1−δ] by choosing δ₂ close to one, while the first sum remains positive and uniformly bounded away from zero over α ε[δ,1−δ]. Thus, for a suitable δ₂, R_{F}(α)−R_{B}(α)>0 for each α ε[δ,1−δ].□

Theorem 2 shows that over a wide range of α values, with suitably selected values of (υ₂ⱼ₋₁², υ₂ⱼ²), the expected total risk for Zcut is less than that for the classification rule derived from Z-tests. The conditions of the theorem are fairly reasonable. The restriction to normally distributed errors is made for convenience since a central limit theorem as in Theorem 1 generally applies in practice. Section 6 will provide several simulations verifying Zcut's better risk performance over the use of Z-tests.

As mentioned earlier, there exists a generalization of the two-group BAM shrinkage plot extended to multiple groups. In one embodiment, a particular group is designated as a baseline, and individual two-dimensional (2-D) plots of pair-wise BAM test statistic values (also called Bayes test statistics) which are group effect test statistics against the designated baseline group are plotted. Accordingly, a number of these 2-D plots are generated based, at least in part, on the total number of groups under study. Note that the posterior variance on these is typically not actually plotted, but rather a cutoff rule based on the posterior variance coalescing to a value of 1 is used as a way to indicate which genes are being significantly turned on or off on these 2-D plots.

For example, and without limitation, if groups A, B, C and D are of interest, and group A is designated to be the baseline, the systems and methods described herein generate the following 3 BAM test statistic by group plots:
BA vs CA
CA vs DA
BA vs DA,
   where the horizontal and vertical axes depict the BAM test statistic values for the group effect of comparing some group relative to the baseline group A. Moreover, as shown in FIG. 3b, points are colored (e.g., magenta for genes that are significantly turned on or off across substantially all groups, and cyan and green for hit-and-run genes) based on whether a gene has a posterior variance coalesced to about 1: something analogous to what is done in the initial two-group shrinkage plot.

This can be further enhanced by choosing one of the generated plots and overlaying primarily significant genes with respect to the omitted group using a different plotting symbol but maintaining the original color scheme. Note that this overlay strategy (or a similar one suitably adapted to the number of groups) is resorted to typically when there are more than 3 groups being studied.

In multigroup problems, typically more than one parameter is estimated for each gene. In the example above, there are 3 parameter estimates of gene effect for each gene. Traditional least square test statistics (Z-test) are typically positively correlated, and this correlation induces errors in classifying genes as being differentially expressed or not (i.e., a mistake made with classifying one of the gene-specific effects, increases the likelihood of making mistakes on the others for that gene). BAM shrinkage reduces this correlation to about zero, and hence mitigates the effects of what is referred to herein as “regression to the mean.” This combined with the original shrinkage of the test statistic point estimates themselves typically yields uniformly superior risk performance in multigroup problems.

As an illustration of this, consider a multigroup analysis of colon cancer data (a problem to be discussed later in this specification). In this context, patterns of differential gene expression across four groups are sought: BSurvivors (the Stage 2 survivors), C (Stage 3), D (Stage 4) and METS (tumors metastasized to the liver). FIGS. 3b and 3c depict a BAM test statistic by group plot and a Z-test by group plot, respectively. In an embodiment shown in FIGS. 3b and 3c, the BSurvivors group is selected as a baseline and two other stages are chosen for plotting (even though the actual analysis was run with all 4 stages). That is, the overlaying plotting option is bypassed for illustration purposes, graphical clarity, and expediency.

FIG. 3b depicts a BAM test statistic (also known as Bayes test statistic) by group plot. Gene-specific BAM test statistic estimates are shown. In this plot, the METS vs BSurvivors effect is plotted against the D vs BSurvivors effect. Clearly, a different plot emerges from the one generated using Z-test. Shrinkage is evident along the coordinate axes (i.e. shrinkage of the corresponding Z-test values shown in FIG. 3c) as well as in the marked non-elliptical shape of the plot (shrinkage of the correlation between gene-specific Z-test values). Coloring here is used to indicate different types of patterns of interest in this multigroup framework: magenta indicates genes significantly turned on or off across the stages and cyan and green indicate hit-and-run genes. Apriori, obvious hit-and-run genes would be ones in cyan and green that hug either coordinate axes (i.e., early or late hit-and-run). These are clearly visible in the Figure. Contrast this with FIG. 3c.

FIG. 3c depicts a Z-test by group plot. The analysis is substantially similar to that presented for FIG. 3b, except the use of standard least square estimates. Clearly hit and run genes (potentially in quadrants 2, 4, 6 and 8) are not visible since they would be ones hugging the coordinate axes in these quadrants. Additionally, note the elliptical shape of the plot indicating positive correlation between the gene-specific effect estimates. Also note that quadrants here were generated by using the simple +/−2 cutoff rule for statistical significance and are numbered going clockwise starting from 1 in the bottom right-hand quadrant.

4. Generating the null: FDRmix. Values estimated from BAM may also be used in a hybrid BH procedure as a method for controlling the FDR. This new model selection procedure is referred to herein as FDRmix. Like the Zcut procedure, FDRmix selects models based on posterior values for μⱼ. In this approach, Tⱼ=E(μⱼ|Y), the posterior mean value for μⱼ, is used as the test statistic in selecting models.

To implement FDRmix, the conditional density for Tⱼ under the null hypothesis μ_{j,0}=0, i.e., F₀(dTⱼ), is determined. Although an exact derivation difficult, an accurate and simple approximation for F₀ may be derived by considering \hat{μ}_{j,n} given earlier in (6). Suppose that the null is true. Although the posterior should identify gene j as having a mean of zero, there will still be a positive posterior probability of a large υ₂ⱼ² and a resulting misclassification. Equation (7) identifies Tⱼ under this scenario. Suppose that the data is balanced with fixed group sizes so that n_{j,1}=N₁ and n_{j,2}=N₂. Then, under the null, conditioning on the event Aⱼ={ν₂ⱼ² is large},

$$( {T}_{j} \vert {A}_{j} , {μ}_{j , 0} = 0 ) \approx \frac{\sqrt{{N}_{2}}}{{\hat{σ}}_{n}} ( {\overline{Y}}_{j , 2} - {\overline{Y}}_{j , 1} ) \approx Normal ( 0 , ( {N}_{1} + {N}_{2} ) / {N}_{1} ) .$$

On the other hand, if the posterior correctly identifies that gene j is not expressing, i.e. that Aⱼ^{c}={ν₂ⱼ²≈0}, then (6) suggests that

$$( {T}_{j} \vert {A}_{j}^{c} , {μ}_{j , 0} = 0 ) \approx \frac{\sqrt{{N}_{2}}}{{\hat{σ}}_{n} ( 1 + 1 / {v}_{2 j}^{2} )} ( {\overline{Y}}_{j , 2} - {\overline{Y}}_{j , 1} )$$

for some small value of υ₂ⱼ². Under the null this also has a normal distribution with mean zero, but unlike the previous case it is not so clear what the theoretical variance is.

These arguments suggest that the null distribution of Tⱼ may be approximated using the two-point normal mixture,
F₀(dTⱼ)≈(1−Π₀)φ(Tⱼ|V₁)+Π₀φ(Tⱼ|V₂),
where φ(·|V) denotes a normal density with mean zero and variance V. It is anticipates that V₂=(N₁+N₂)/N₁ but the values for V₁ and Π₀=Pr{Aⱼ|μ_{j,0}=0} are unknown. All of these values, however, may be estimated by fitting a two-point normal mixture to simulated data. Thus, to estimate V₁, V₂ and Π₀ data from the model (4) may be simulated, where μ_{j,0}=0 for all j=1, . . . , p (typically p is chosen to be some large number, e.g., 25000). Notice this simulation requires little, if anything, more than knowing the sample sizes N₁ and N₂ and the value for σ₀², which may be estimated accurately from the original data. The BAM systems and methods described herein are then applied to the simulated data and a two-point mixture model is fitted to the averaged values for μⱼ collected from the Gibbs output. The results from fitting the mixture can now be used to derive p-values which are then analyzed in the usual way by the BH method to determine which hypothesis to reject. To compute the p-values suppose that Tⱼ^{o} is the estimated value for E(μⱼ|Y) from the original (non-simulated) data. Then its p-value Pⱼ may be approximated by
Pⱼ=2Pr{|Tⱼ^{o}|<Tⱼ|μ_{j,0}=0}≈2(1−Π₀Φ(|Tⱼ^{o}|/\sqrt{V₁})−(1−Π₀)Φ(|Tⱼ^{o}|/\sqrt{V₂})),
where Φ(·) denotes a standard normal cdf and the expectation on the left is over the null.

5. Comparison procedures. BAM was tested against several different model selection procedures. These included what are called “Bayes Exch,” “Z-test,” “Bonf,” and “BH.” Below is a brief description of these methods.

5.1. Z-test. Here genes are identified by Z-test statistics defined earlier in (9). Z-test identifies gene j as expressing if |Z_{j,n}|≧z_{α/2}.

5.2. Bonf. The Bonf procedure is a Bonferroni correction to Z-test. Thus gene j is identified as expressing if |Z_{j,n}|≧z_{α/(2p)}.

5.3. BH. The BH procedure was also applied. The p-values used were based on the test statistics Z_{j,n} under a two-sided test. Thus, if
Pⱼ=2Pr{Normal(0, 1)>|Z_{j,n}|},
then gene j is considered expressing if Pⱼ<=P₍ₖ₎, where P₍ₖ₎ is the k-th ordered p-value where k=max{j: Pⱼ<jα/p} and α>0 is some pre-chosen target FDR. Although the original BH procedure was designed to control the expected FDR assuming independent null hypotheses, it has been shown that the method applies under certain types of dependencies. For example, the method computed from dependent Z-tests such as Z_{j,n} will control the expected FDR if applied to those Z_{j,n} for which the null is true. Thus, when many nulls are true, this approximately controls the expected FDR.

5.4. Bayes Exch. Also implemented was a simple Bayesian exchangeable model. To model (4), the data Y_{j,k,l} is replaced with sufficient statistics \overline{Y}_{j,2}−\overline{Y}_{j,1}. Conditional on \hat{σ}ₙ² this generally yields a Normal(μⱼ, \hat{σ}ₙ²(1/n_{j,1}+1/n_{j,2})) distribution. Thus, to identify genes the empirical hierarchical model was used
(\overline{Y}_{j,2}−\overline{Y}_{j,1}|μⱼ,\hat{σ}ₙ²)˜Normal(μⱼ,\hat{σ}ₙ²(1/n_{j,1}+1/n_{j,2}))
(μⱼ|σ₀²)˜Normal(0,τ₀²)
(τ₀⁻²|t₁,t₂)·Gamma(t₁,t₂),
where t₁=t₂=0.0001 was selected to ensure a non-informative prior for τ₀². This extends the models considered by other researchers, by allowing for a hyperprior on τ₀².

Observe that given the data Y the hyperparameter τ₀² and the estimate \hat{σ}ₙ²,
(μⱼ|Y,\hat{σ}ₙ²,τ₀²·Normal(τ₀²(\overline{Y}_{j,2}−\overline{Y}_{j,1})/(S_{j,n}²+τ₀²),S_{j,n}²τ₀²/(S_{j,n}²+τ₀²)),
where S_{n,n}²=\hat{σ}ₙ²(1/n_{j,1}+1/n_{j,2}). Thus, a reasonable procedure identifies gene j as expressing if
|\overline{Y}_{j,2}−\overline{Y}_{j,1}|≧z_{α/2}S_{j,n}\sqrt{1+S_{j,n}²/\hat{τ}}₀², (10)
where \hat{τ}₀² is some estimate for τ₀². In all examples considered, \hat{τ}₀²=E(τ₀²|Y,\hat{σ}ₙ²).

Notice that when \hat{τ}₀²→∞ the limit of the test (10) corresponds to a standard Z-test. However, whenever \hat{τ}₀²<∞, the Bayesian test (10) is more conservative.

6. Simulations. To assess the procedures, they were tested simulated data from the ANOVA model (4). Means for genes were set to have a group mean separation of μ_{j,0}=1.5 and μ_{j,0}=0 for 10% and 90% of parameters respectively. This corresponds to a model in which 10% of genes are expressing and represents a fairly realistic scenario for microarray data. For convenience θ_{j,0} were set to zero for all j. Thus, gene simulation model was characterized by
Y_{j,k,l}=μ_{j,0}I{l=2}+ε_{j,k,l}, j=1, . . . p, k=1, . . . , n_{j,l}, l=1,2,
where ε_{j,k,l} were taken to be i.i.d N(0, σ₀²) variables. For the variance, σ₀² were set to one. Group sizes were fixed at n_{j,1}=n_{j,2}=5 and number of genes at p=25000 (the simulation reported in Table 1 and FIG. 1 used the same configuration as above but with p=10000).

All model estimators reported were based on a range of preset α levels. For Zcut, Bayes Exch, Z-test and Bonf, a preset α value meant that the corresponding test was computed based on the appropriate quantile for a standard normal (for example, Z-test was computed using z_{α/2} as a cutoff while Bonf used z_{α/(2p)}). For FDRmix and BH the α value used was the target FDR. To fairly compare the procedures under the same α values the total number of misclassified observations, TotalMiss, discussed in the Introduction, were recorded. Also recorded were the FDR, FNR and the Type-I and Type-II error rates.

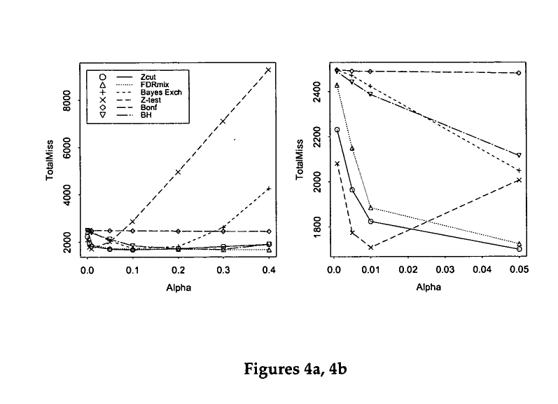

The results of the simulations are reported in Table 2 and FIG. 4. From these the following general observations may be made:

1. Z-test has the best total risk performance for small a values, but its good risk behavior is only local. For example, once a increases the value for TotalMiss increases rapidly and procedures like Zcut and FDRmix quickly have superior risk performance. Furthermore, if a becomes very small, its TotalMiss values also shoot up. Thus, trying

TABLE 1
Results from gene simulation model (α = 0.05).

| | Detected | TotalMiss | FDR | FNR | Type-I | Type-II |
|---|---|---|---|---|---|---|
| Zcut | 481 | 675 | 0.162 | 0.063 | 0.009 | 0.597 |
| FDRmix | 416 | 702 | 0.142 | 0.067 | 0.007 | 0.643 |
| Z-test | 1106 | 792 | 0.406 | 0.039 | 0.049 | 0.343 |
| BH | 148 | 862 | 0.034 | 0.087 | 0.001 | 0.857 |

TABLE 2
Gene simulation model p = 25000 with 10% of genes expressing.
Estimators based on α levels of 0.05, 0.01, 0.001 respectively.

| | Detected | TotalMiss | FDR | FNR | Type-I | Type-II |
|---|---|---|---|---|---|---|
| Zcut | 1286 | 1700 | 0.189 | 0.061 | 0.011 | 0.583 |
| | 822 | 1824 | 0.088 | 0.072 | 0.003 | 0.700 |
| | 281 | 2231 | 0.021 | 0.090 | 0.000 | 0.890 |
| FDRmix | 1148 | 1724 | 0.162 | 0.064 | 0.008 | 0.615 |
| | 719 | 1885 | 0.072 | 0.076 | 0.002 | 0.733 |
| | 72 | 2430 | 0.014 | 0.097 | 0.000 | 0.972 |
| Bayes Exch | 498 | 2048 | 0.046 | 0.083 | 0.001 | 0.810 |
| | 78 | 2424 | 0.013 | 0.097 | 0.000 | 0.969 |
| | 3 | 2497 | 0.000 | 0.099 | 0.000 | 0.999 |
| Z-test | 2808 | 2006 | 0.412 | 0.038 | 0.051 | 0.340 |
| | 1245 | 1709 | 0.182 | 0.062 | 0.010 | 0.593 |
| | 453 | 2081 | 0.038 | 0.084 | 0.001 | 0.826 |
| Bonf | 18 | 2482 | 0.000 | 0.099 | 0.000 | 0.993 |
| | 10 | 2490 | 0.000 | 0.099 | 0.000 | 0.996 |
| | 2 | 2498 | 0.000 | 0.099 | 0.000 | 0.999 |
| BH | 415 | 2115 | 0.036 | 0.085 | 0.001 | 0.840 |
| | 116 | 2388 | 0.017 | 0.096 | 0.000 | 0.954 |
| | 11 | 2489 | 0.000 | 0.099 | 0.000 | 0.996 |

to find the small region of a values where Z-test does well is a tricky proposition. No theory exists to select this region, which makes Ztest unreliable. Overall, Zcut and FDRmix have the best total risk behavior.

For small to moderate α values Zcut is better, while FDRmix is better for large α. BH has poorer performance for small to moderate α, only approaching the same performance as Zcut and FDRmix for α as large as 0.2. This confirms its inability to simultaneously optimize the FDR and FNR. Bayes Exch tracks BH in terms of total risk over small to moderately large α.
2. FDRmix does not achieve target α values but does further reduce the FDR for Zcut as designed. The FDR for BH is better, but is generally smaller than its target value. When α becomes small its FDR drops to zero and it becomes too conservative.
3. Among all the procedures tested Zcut has Type-II error rates closest to those observed for Z-test.
4. The worst procedure overall is Bonf whose TotalMiss plot is flat. Alternatives to Bonferroni corrections have been attempted for microarrays. These often involve nonparametric stepdown adjustments to raw p-values using permutation distributions of the test statistics. Modest power gains have been seen but coarseness of the permutation distributions limit the usefulness of these approaches to situations where a large enough number of samples are available. To illustrate, an adjusted p-value method was applied, and it was found that with α equal to 0.05, 0.01 and 0.001, 18, 10 and 2 significant genes were found, respectively. This performance mirrors that of the Bonferonni method. Since step-down adjustment methods also attempt to control family wise error rates, it's not surprising at how conservative they were found to be.

Interestingly, although FIGS. 4a-4b show that Z-test may be quite volatile near its optimal a value, the plots suggest that Z-test nevertheless outperforms Zcut for smaller α values. Thus, Z-test's volatility may not be a problem as long as one adopts the strategy of choosing a small α value. In fact, this can be a bad strategy as the TotalMiss values for Z-test can sometimes rise rapidly even at near zero values for α. To illustrate this behavior the signal to noise ratio of the previous simulation was reduced by setting μ_{j,0}=0.5 for the 10% of genes that were expressing. All other values of the simulation were kept the essentially the same.

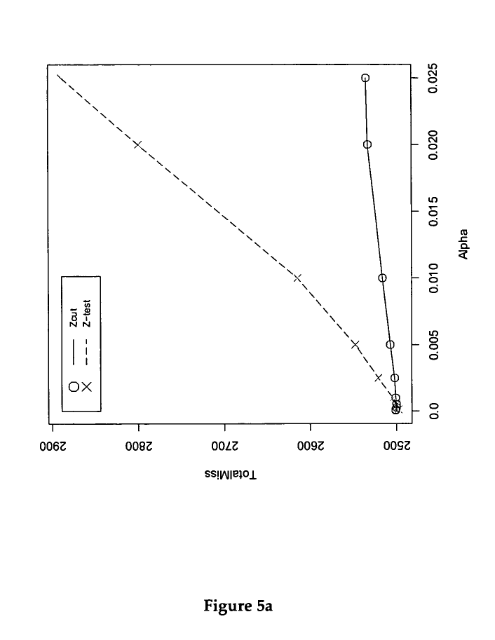

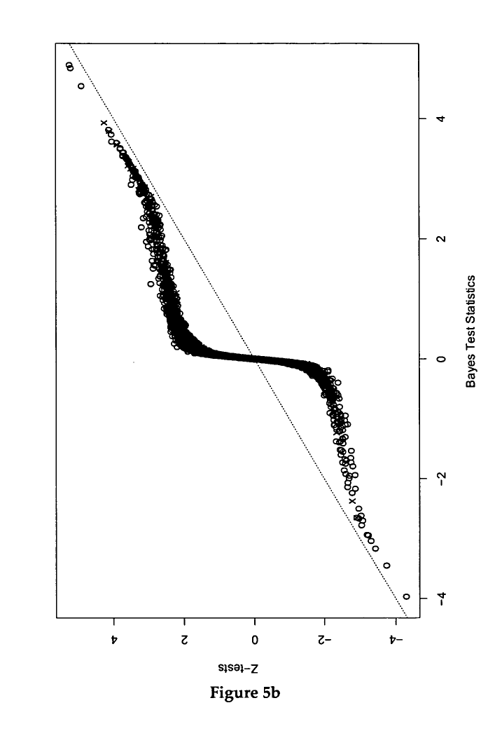

FIG. 5a records the TotalMiss values for Z-test and Zcut from the analysis by depicting the total number of misclassified observations from the gene simulation model used in FIG. 4, but with smaller signal-to-noise ratio. The plot clearly shows that Zcut has superior risk performance over Z-test even at near zero a values. Notice also how the TotalMiss values for Z-test increase rapidly due to overfitting, while the risk performance for Zcut stabilizes. The better risk performance by Zcut is due, at least in part, to its adaptive shrinkage ability as predicted by Theorem 2.

FIG. 5b shows the extent to which BAM is able to shrink test statistics compared to the Z-test. The figure plots the BAM test statistics against Z-tests similar to FIG. 3a. An 0 denotes Zcut, and “x” denotes Z-test. It is instructive to compare this to FIG. 3a to see how much shinkage is actually going on.

6.1. Unequal Variances: Poisson Gene Simulation Model. Untransformed gene expression data often fail to satisfy the assumptions of an equal variance model. A pattern often seen is that the variance for a gene expression is related to its mean expression. Often the relationship is linear, but sometimes it may be quite complex, such as in the case of colon cancer (Section 7). To deal with these problems, it is generally recommended that the data be transformed by a variance stabilizing transformation.

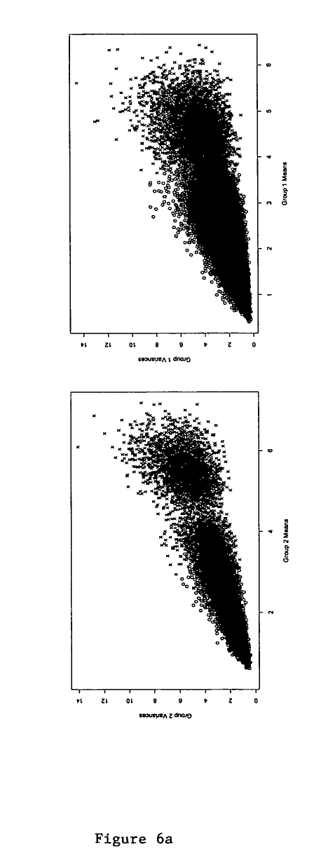

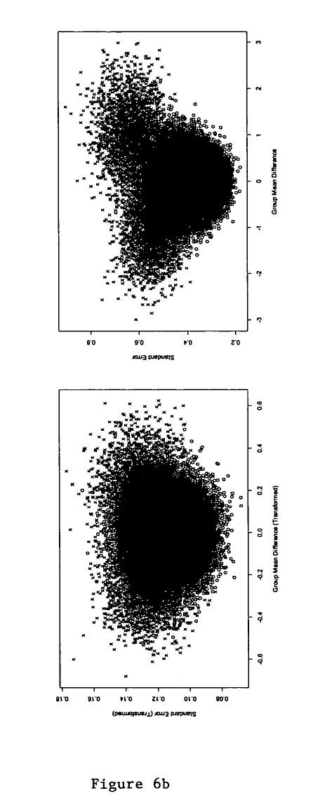

To study how well the BAM systems and methods described herein perform under the scenario wherein variances are proportional to the mean, and also to see how robust they are in light of an assumption of normality, data from the Poisson model
Y_{j,k,l}=ξ_{j,k,l}+ε_{j,k,l}, j=1, . . . , p, k=1, . . . , n_{j,l}, l=1,2,
were simulated, where ξ_{j,k,l} are independent Poisson(μ_{j,l}) variables, independent of ε_{j,k,l}, the N(0, σ₀²) measurement errors. The variances σ₀²=0.01 were set to a small value. This slightly smooths the data, although at the same time the variance for a gene expression will be proportional to its mean. FIG. 6(a) shows gene mean values \overline{Y}_{j,l} plotted against gene sample variances \hat{σ}_{j,l}². The left-hand side is group l=1, while the right-hand side is group l=2.

Expressing genes are identified by a cross, while non-expressing genes by a circle. Gene group sizes n_{j,1}=21 and n_{j,2}=34 were used, and the number of genes was set at p=60000. These sample sizes were selected to match those of the colon cancer data set. About 90% of the genes were set to have equal means over both groups, so that for these genes μ_{j,1}=μⱼ, where μⱼ was drawn randomly from a uniform distribution on (1, 3). For the remaining 10% of the genes (the expressors), the group one means μ_{j,1} were sampled independently from a uniform distribution on (4,5). Then the group two means μ_{j,2} were set to this value, incrementing by either +1 or by −1 randomly with equal probability. This corresponds to genes for the treatment turning on or off.

The better performance of the methods on the untransformed data can be explained by FIG. 6(b), which shows group mean differences \overline{Y}_{j,2}−\overline{Y}_{j,1} versus standard errors. The left-hand side includes the untransformed data, whereas the right-hand side is computed from the variance-stabilized data. On the left-hand side the untransformed data is plotted for the group mean difference \overline{Y}_{j,2}−\overline{Y}_{j,1} for each gene versus the corresponding standard error (\hat{σ}_{j,1}²/n_{j,1}+\hat{σ}_{j,2}²/n_{j,2})^{1/2}, where

$${\hat{σ}}_{j , l}^{2} = \frac{1}{{n}_{j , l} - 1} {\sum }_{k = 1}^{{n}_{j , l}} {( {Y}_{j , k , l} - {\overline{Y}}_{j , l} )}^{2} ,$$

is the sample variance for gene j over group l.

It is seen that even though the standard error is not constant over the group mean differences (compare this to the square-root transformed plot on the right-hand side), its value still remains fairly constant, only becoming elevated for large group mean differences, where it remains relatively small in magnitude compared to the mean effect. Thus, even though in the untransformed data the gene sample variances are proportional to their means (FIG. 6(a)), once the sample size is taken into account when computing the standard error, the effect of unequal variances gets washed out. It seems better to simply not transform the data, since a side effect is that means are compressed, leading to smaller models.

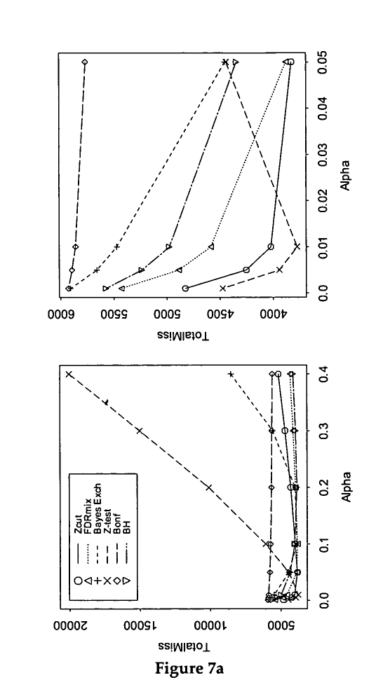

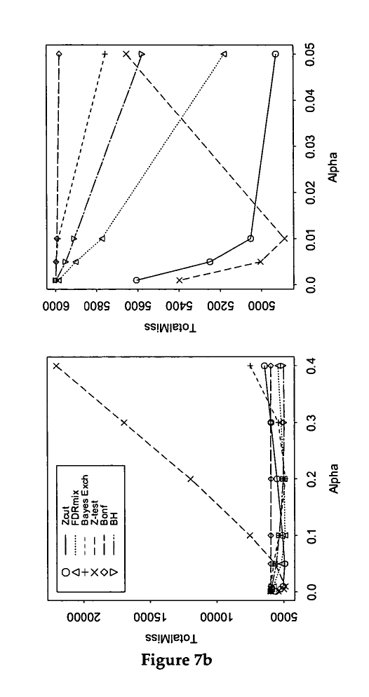

The results of the Poisson simulation are reported in FIG. 7, which plots the total number of misclassified observations as α is varied. FIG. 7(a) is from the untransformed data, while FIG. 7(b) are results from the data under a square-root variance stabilizing transformation. The following conclusions may be drawn:
1. The variance stabilizing transform reduces the size of models across essentially all estimators with fewer genes being identified as expressing. In essentially all cases the value for TotalMiss increases under the transformation.
2. Estimators generally exhibit the same patterns of behavior as in the previous simulation.

6.2. Dependence: Correlations Between Genes. The simulations up to this point have generally assumed that genes are independent, but in practice it is more realistic to expect gene expressions to be correlated. A gene's expression across different samples may be safely assumed to be independent in experimental designs in which for a fixed j, the values of Y_{j,k,l} represent expressions measured over different tissue types l for different samples k. Within a given sample k, however, different genes can cluster into small groups—often a manifestation of gene proximities along biological pathways. This has been termed “clumpy dependence,” and is the most likely scenario of dependence for these types of experimental designs. To study how the procedures perform under this form of dependence data were simulated according to the model Y_{j,k,l}=μ_{j,0}I{l=2}+ζ_{mj,k,l}+ε_{j,k,l}, j=1, . . . , p, k=1, . . . , n_{j,l}, l=1,2

where ε_{j,k,l} are i.i.d N(0, σ₀²) measurement errors, independent of ζ_{mj,k,l}. The same dimensions and group sizes were used as in the earlier set of simulations: p=25000, n_{j,1}=n_{j,2}=5. Variables ζ_{mj,k,l} were introduced to induce dependence amongst genes into small “blocks.” Here, mⱼ=[j/B], where B=50 and [j/50] represents the first integer greater than or equal to j/50. Thus, for each fixed k and l, there were 500 variables ζ_{1,k,l}, . . . , ζ_{500,k,l}. These each induce a block of B dependent observations. For example, “block m” includes those Y_{j,k,l} where [j/B]=m, which are dependent since they share the common value ζ_{m,k,l}. Variables across different values for k and l were assumed independent. That is, ζ_{j,k,l} was independent of ζ_{j,k′,l′} if k≠k′ or l≠l′. This ensured that a gene's expression was independent across samples.

TABLE 3
Dependent gene simulation model. Estimators based on α levels
of 0.05, 0.01, 0.001.

| | Detected | TotalMiss | FDR | FNR | Type-I | Type-II |
|---|---|---|---|---|---|---|
| Zcut | 1299 | 1702 | 0.191 | 0.061 | 0.011 | 0.581 |
| | 807 | 1868 | 0.109 | 0.074 | 0.004 | 0.712 |
| | 286 | 2230 | 0.029 | 0.089 | 0.000 | 0.889 |
| FDRmix | 1146 | 1738 | 0.166 | 0.065 | 0.008 | 0.618 |
| | 686 | 1939 | 0.094 | 0.077 | 0.003 | 0.751 |
| | 33 | 2467 | 0.000 | 0.099 | 0.000 | 0.987 |
| Z-test | 2762 | 1979 | 0.404 | 0.039 | 0.049 | 0.343 |
| | 1269 | 1698 | 0.184 | 0.062 | 0.010 | 0.586 |
| | 473 | 2078 | 0.055 | 0.084 | 0.001 | 0.821 |
| BH | 403 | 2129 | 0.029 | 0.086 | 0.001 | 0.845 |
| | 66 | 2435 | 0.000 | 0.098 | 0.000 | 0.974 |
| | 4 | 2496 | 0.000 | 0.099 | 0.000 | 0.999 |

The first 2500 (10%) of genes were set to have non-zero μ_{j,0} coefficients and the remaining 90% to have zero coefficients. This ensured that blocks were made up entirely of expressing genes or entirely of non-expressing genes. All ζ_{mj,k,l} variables were drawn from a Normal(0, η₀²) distribution.

The values η₀²=19 and σ₀²=1 were selected to induce a correlation of ρ₀=0.95 between observations within the same block. So that the signal-to-noise ratio would be similar to the earlier simulation, μ_{j,0}=1.5/\sqrt{1−ρ₀} were selected for the expressing genes.

Table 3 presents the results in a style similar to Table 2 for direct comparison. Values reported in Table 3 were obtained by averaging over 100 independent simulations. For brevity, only the results for Zcut, FDRmix, Zcut and BH are reported. The values for TotalMiss and Detected were rounded to the nearest integer.

Comparing Table 3 to Table 2 the following obersvations may be made:
1. The FDR procedures, FDRmix and BH, start to break down as α decreases. The number of detected genes drops rapidly and the FDR becomes essentially zero. FDR procedures will have high variability in dependence scenarios and are less reliable for low α than for high α. Techniques are available for correcting FDR methods under dependence.
2. On the other hand, Zcut and Z-test have performance measurements similar to those seen in Table 2. Overall, clumpy dependence seems to have a minimal effect on them. This is partly explained in that these procedures classify genes based on test statistics that are affected by dependence mainly through \hat{σ}ₙ². As long as block sizes B are relatively small compared to p (a likely scenario with microarray data), the effect of dependence is marginal since \hat{σ}ₙ², is a robust estimator for the variance, \hat{σ}ₙ²+η₀².

7. Metastatic signatures for colon cancer. A practical illustration of the BAM systems and methods is, as discussed above, detection of a metastatic gene expression signature for colon cancer. This problem is of significant medical importance. Colorectal cancer is the second leading cause of cancer mortality in the adult American population, accounting for 140,000 new cases annually and 60,000 deaths. The current clinical classification of colon cancer is based on the anatomic extent of the disease at the time of resection. It is known that this classification scheme is highly imperfect in reflecting the actual underlying molecular determinants of colon cancer behavior. For instance, upwards of 20% of patients whose cancers metastasize to the liver are not given life saving adjuvant chemotherapy based on the current clinical staging system. Thus, there is an important need for the identification of a molecular signature that will identify tumors that metastasize. In addition, this signature will no doubt suggest new targets for the development of novel therapies.

The gene expression considered herein is from the Ireland Cancer Center at Case Western Reserve University. The Center has collected a large database of gene arrays on liver metastasized colon cancers (METS), modified Duke's B1 and B2 survivors (B-survivors) as expressed by the Astler-Coller-Dukes staging system (Cohen et al., 1997), and normal colon tissue samples. The B-survivors represent an intermediate stage of tumor development with metastasis to the liver being seen as the disease worsens. As of February, 2002, there were 76 total samples available for analysis made up of 34 METS, 21 B-survivors and 21 normals.

The gene arrays used in compiling the database were EOS Biotechnology 59K-on-one chips, which were Affymetrix chips modified for using a smaller subset of eight more sensitive probes for each gene than do the original Affy chips which use 20 probes per gene. An advantage of such a system is the ability to assay many more genes on each chip. In fact, yields of up to about 60,000 pieces of genetic information are capable of being processed on each chip. Generally, the genes are treated as independent, realizing that there is still an open debate as to the total number of actual genes in the human genome.

The analysis was based on the data for the liver METS and B-survivor tissue samples (normal tissue samples were excluded as a primary interest was understanding cancer evolving from the intermediate B-survivor stage). Using the earlier notation, group sizes were n_{j,1}=21 for B-survivors (the control group) and n_{j,2}=34 for the liver METS (treatment group). In total there were nⱼ=55 samples for each of p=59341 probe sets.

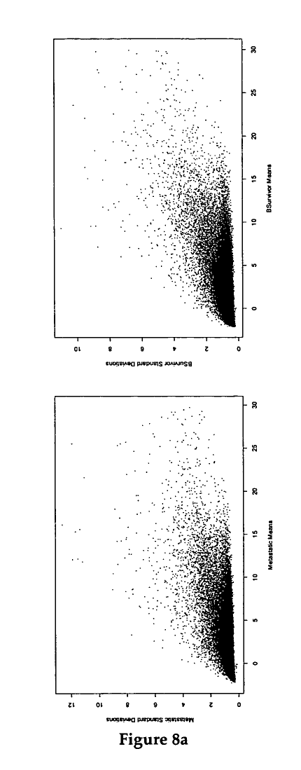

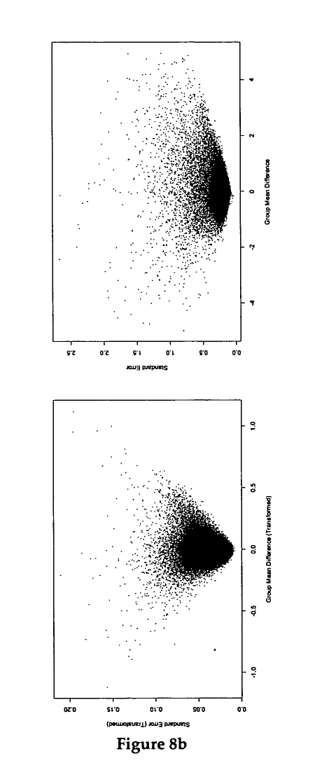

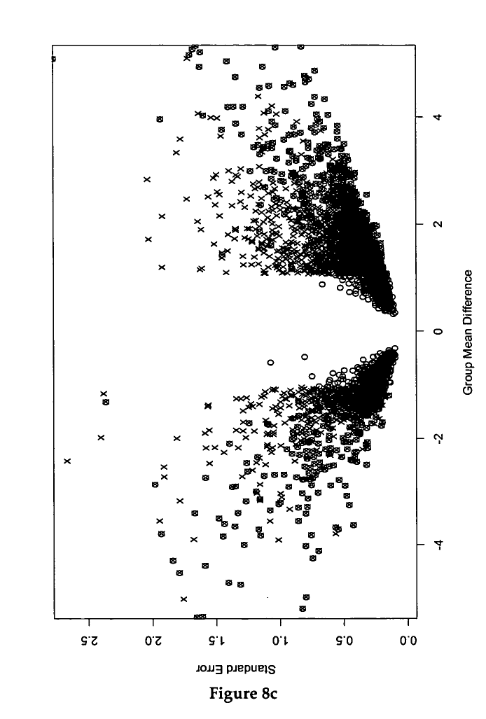

FIGS. 8(a) and 8(b) plot different summary values for the data. FIG. 8(a) shows the gene mean values \overline{Y}_{j,1} versus gene standard deviations \hat{σ}_{j,1} from colon cancer data. Primarily mean values less than about 30 are plotted, to help zoom in plot. Here the mean for a specific gene is plotted against its standard deviation in order to identify any potential mean-variance relationship. The figure reveals a pattern that at first glance appears to be linear. However, careful inspection shows that although standard deviations are generally increasing with the mean expression for a gene, one can also see a broad strip of values where standard deviations are essentially constant. Thus, the pattern here is one of overdispersed variances.

This raises the question of how to handle the unequal variances. One approach includes trying different transformations in the hope of stabilizing the variance. Consider the right-hand side of FIG. 8(b), which is the result of the log-transformation log(Y_{j,k,l}+1+Δ), where Δ=|min{Y_{j,k,l}}|; the constant 1+Δ has been added to ensure that gene expressions were strictly larger than one so that logs for small values were not unduly inflated. The figure shows the group mean differences \overline{Y}_{j,2}−\overline{Y}_{j,1} versus standard errors. The left-hand side is computed from untransformed data, while the right-hand side is computed from the log-transformed data. For untransformed data, mean differences less than about 5 in absolute value are plotted to help zoom in plot. As may be seen, the transformation has helped to stabilize the standard error when compared to the mean differences between the two tissue groups.

As was discussed in Section 6.1, the stability of the standard error is an important criterion for assessing validity of inference based on an equal variance model. However, as also discussed, the gain in stabilizing standard errors by a transformation may sometimes be offset by the manner in which mean values are nonlinearly transformed. Evidence that this is happening for the data can be seen from FIG. 8(c), which shows genes found significant using Zcut with α=0.0005. Crosses denote genes detected from untransformed data, while circles are from log-transformed data. In other words, in the figure are plotted the genes detected by Zcut on both the untransformed and log-transformed data using the two group ANOVA model of Section 3 (Zcut based on an α=0.0005 cutoff criteria). As can be seen, the log-transform appears to be encouraging Zcut to pick genes with very small mean differences; thus, the effect of the transform is to overly enhance the signal-to-noise ratio for these genes. Such problems with the log-transform have been noticed elsewhere, and some researchers argue against the use of log-transforms in analysis of gene expression data.

However, some form of variance stabilization is called for here as the left-hand side of FIG. 8(b) reveals that the standard errors for the untransformed data are quite variable, certainly more than what was seen in the Poisson simulations of Section 6.1. Global transformations to the data lead to undesirable results. Therefore, the classical method of weighted regression was relied on to deal with heteroscedasticity. In this approach, genes were grouped into C=2 clusters, depending on whether the standard error for a gene was less than or greater than about the 99th percentile for standard errors. This allows treatment of the small group of genes with very high variability differently from the remaining genes. For each group, gene expressions Y_{j,k,l} were then rescaled by dividing by the square root of the group mean-square error (the unbiased estimator for the variance). The BAM systems and methods described herein were then applied to the newly rescaled data. It should be noted that this method may be applied for any number of clusters C, although it is generally recommended that the data undergo as little transformation as possible in trying to reduce variability.

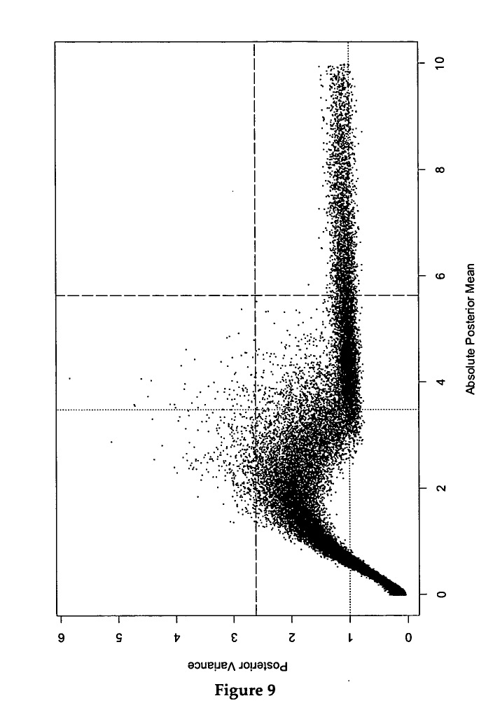

FIG. 9 depicts estimated values for |E(μⱼ|Y)| versus Var(μⱼ|Y) using weighted regression. Values for |E(μⱼ|Y)| less than about 10 are plotted to help zoom in plot. The figure contains the shrinkage plot of the posterior absolute means and posterior variances for μⱼ from the Gibbs output using the weighted regression approach. Once again a variance of one is represented by a thin dashed horizontal line, while the thick dashed horizontal line represents the asymptotic value nⱼ/n_{j,1}=55/21.

The two vertical lines in the figure represent the 100×(1−α/2) percentile from the appropriate normal distributions for α=0.0005. The thin dashed line denotes the uncorrected N(0, 1) distribution, while the thick dashed line is from the adjusted N(0, nⱼ/n_{j,1}) distribution. The value for α=0.0005, which we used for the cutoff value in the analysis, was chosen here by eye-balling FIG. 9 and choosing the vertical line that makes almost all observations to its right have posterior variance of roughly one. This removes intermediate values that could inflate the FDR; the same technique was used to select the value of α=0.0005 in FIG. 8(c).

TABLE 4
Number of colon cancer genes detected using
weighted regression approach.

| Zcut | FDRmix | Bayes Exch | Z-test | Bonf | BH |
|---|---|---|---|---|---|
| 3743 | 3334 | 3433 | 4576 | 1369 | 2960 |

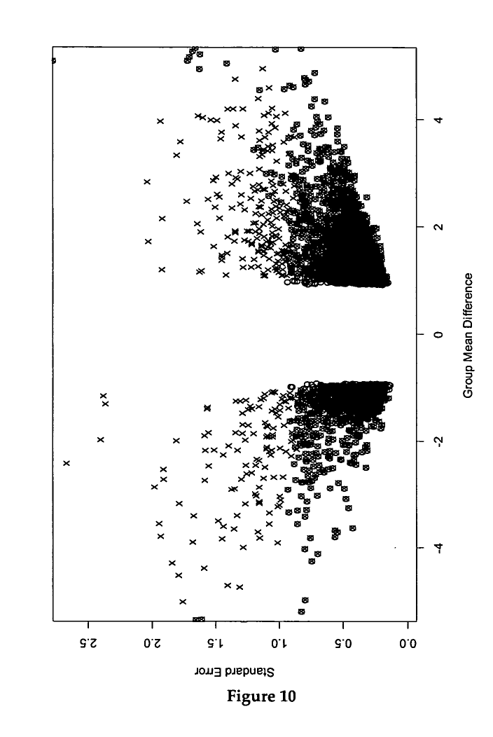

FIG. 10 shows genes found significant using Zcut with α=0.0005. Crosses denote genes detected from original data, while circles denote using a weighted regression approach. Group mean differences and standard errors plotted are computed from the original data, similar to FIG. 8(c). Mean differences less than about 5 in absolute value are plotted to help zoom in plot. The figure shows the genes identified by Zcut from the original data and for the new data formed by the weighted regression approach. Observe how the weighting allows Zcut to pick up moderate to high expression genes as desired, while allowing genes with small expressions but high standard errors to be dropped off. Zcut is also able to pick up genes with smaller expressions which have small standard errors, but it does not pick up very small mean expression values, as observed with the log-transform. Thus, the new Zcut is more flexible, but there is still a large overlap in detected genes as desired. Of the 3743 genes identified by Zcut over the weighted data, 2864 (76.5%) were also identified over the original data. Table 4 records the number of genes detected by each of the methods for the weighted data (a value of 0.0005).

Interestingly, substantially all of the genes picked by BH are contained

TABLE 5
Grouping of genes found by Zcut and not by BH by
biological function

| Functional Group | Number of genes |
|---|---|
| Transcription factor | 3 |
| DNA binding | 1 |
| Cell adhesion | 3 |
| G-protein signaling | 2 |
| STAT cascade | 3 |
| TGF receptor ligand | 2 |
| Growth factor receptor ligand | 1 |
| Oncogene | 2 |
| Intracellular signaling | 4 |
| Signal transduction | 6 |
| Cell communication | 15 |

in the list of genes picked by Zcut. These are clearly the genes showing large differential expression between B-survivors and METS. It is informative to look at the list of non-overlapping genes between these two methods. There were 783 genes in this non-overlapping set. This number was further reduced to 779 after removing genes indicating potential liver or spleen contamination. Following this, two quality control analyses were run independently to assess differences in samples (some genes were prepared in San Francisco and others in Cleveland). Genes showing differences in sites were excluded, as where genes showing variability between tumors on the same patient.

In the end, there were 476 genes in this non-overlapping set. Of these, 193 were stripped away from further analysis since they were expressed sequence tags (EST's). This left 283 genes from which EOS probe sets were mapped to Affymetrix U133A and U133B human gene chips. Once this conversion was done, the information was entered into GeneSpring, a software used to extract functional information about genes printed on Affymetrix chips. The remaining genes where then categorized into different functional groups. The findings were quite interesting. Table 5 provides a breakdown for groupings which represent important functional pathway steps identified in the metastasis process. These include genes which are transcription factors and genes involved in DNA binding, cell adhesion and various signaling, cell communication or cascade pathways. In fact, some of the cascade pathways have been identified as potential sources of possible gene-to-gene interactions due to dimerizations produced during a particular step in a cascade.

8. Discussion. It has been shown how to cast the problem of looking for differentially expressed genes in microarrays as a high dimensional subset selection problem using BAM, a powerful new Bayesian model selection technique. Although the BAM systems and methods were considered here in detail in the context of the two-way ANOVA model, the method permits extensions to more than two groups. Extensions to ANCOVA models are also possible for use in study designs where gene expressions are measured under various experimental conditions.

An important feature of BAM is that it can be used differently depending upon the need of the user. For example, Zcut, a model estimator computed from posterior means, amounts to a weighted combination of ridge regression estimators and strikes a balance between low FDR values and higher power. If the FDR is of greater importance, this disclosure has provided a novel technique for estimating the distribution of gene effects under the null hypothesis using a two-component mixture model, leading to what has been termed herein as the FDRmix procedure. The shrinkage effect of BAM provides the power gains seen for the model estimators herein. BAM adaptively shrinks test statistics for intermediate and small sized effects, whereas larger effects yield test statistics similar to Z-test. This is the reason why even though more gene effects are detected as significant using the systems and methods described herein than, say, BH, a source of conservatism is built-in minimizing the potential of too many falsely rejected null hypotheses.

The significance of the increased power was evident attempting to determine a metastatic genetic signature of colon cancer. Known to be a very complex disease process, Zcut detected over 700 more significant genes than BH did. Many of these genes in fact turned out to be potentially implicated in the metastasis of B-Survivor solid tumors from the colon to the liver. It was interesting that no currently known genes with obvious involvement in colon cancer metastasis were part of the non-overlapping list. The implications of this work becomes clearer by noting that omitting potentially important genes at the level of initial filtering may lead to further derived discriminant rules based on these filtered subsets of genes that may end up leaving out valuable information.

The colon cancer example also illustrated the difficulties in finding global variance stabilizing transformations for the data. Complex non-linear relationships between the gene effect variances and means can result in an adverse affect on the mean when applying such transformations. This was further illustrated via Poisson simulations. As an alternative, a weighted regression approach was presented. This approach is not limited to the BAM technique and can be used with standard methods as well.

8.1. Extensions to More than 2 Groups. Model (4) can be easily extended to the case involving more than two groups. By way of illustration, an outline of the case involving three groups is now presented. As before, assume that group l=1 is the control group. Groups l=2 and l=3 represent two distinct treatments. If Y_{j,k,l} are the expressions, then testing for treatment effect can be formulated using the ANOVA model,
Y_{j,k,l}=θⱼ+μ_{j,2}I{l=2}+μ_{j,3}I{l=3}+ε_{j,k,l},
where j=1, . . . , p, k=1, . . . , n_{j,l}, l=1,2,3, and ε_{j,k,l} are iid N(0, σ²). Testing whether genes differ in groups 2 and 3 from the control corresponds to testing whether μ_{j,2} and μ_{j,3} differ from zero. As before many of the θⱼ parameters will be non-zero; therefore, the dimension of the parameter space is reduced from 3p to 2p by centering the responses. Applying the appropriate resealing as part of the pre-processing of the data, Y_{j,k,l} is replaced with

$${\tilde{Y}}_{j , k , l} = ( {Y}_{j , k , l} - {\overline{Y}}_{j , 1} ) \times \sqrt{n / {\hat{σ}}_{n}^{2} ,}$$

$$where$$

$${\hat{σ}}_{n}^{2} = \frac{1}{n - 3 p} {\sum }_{j , k , l}^{} {( {Y}_{j , k , l} - {\overline{Y}}_{j , 1} I \{ l = 1 \} - {\overline{Y}}_{j , 2} I \{ l = 2 \} - {\overline{Y}}_{j , 3} I \{ l = 3 \} )}^{2} ,$$

and \overline{Y}_{j,l} is the mean for gene j over group l.

8.2. Differing measurement errors: heteroscedasticity. One can also extend the ANOVA model (4) to handle differing measurement error variances σⱼ² for j=1, . . . , p. Suppose that genes can be bunched into C clusters based on their variances. Let iⱼ ε {1, . . . , C} indicate the variance cluster gene j belongs to. Now modify (4) by replacing ε_{j,k,l} with variables, say \tilde{ε}_{j,k,l}, where \tilde{ε}_{j,k,l} are independent with a N(0, σᵢ_{ⱼ}²) distribution. To handle heteroscedastic variances a weighted regression is applied by reweighting observations by the inverse of their standard deviation. Thus, (4) can now be written as
σᵢ_{ⱼ}^{-1/2}Y_{j,k,l}=θⱼ*+μⱼ*I{l=2}+ε_{j,k,l}, j=1, . . . , p, k=1, . . . , n_{j,l}, l=1,2,
where θⱼ*=σᵢ_{ⱼ}^{-1/2}θⱼ and μⱼ*=σᵢ_{ⱼ}^{-1/2}μⱼ denote the new parameters. The C distinct variances σ₁², . . . ,σ_{C}² are unknown, but they can be estimated accurately when p is large, and thus we can accurately approximate σᵢ_{ⱼ}^{-1/2}Y_{j,k,l}. Hence, if \hat{σ}_{c}² is our estimator for σ_{c}² (we use the usual unbiased estimator) simply replace the data Y_{j,k,l} with rescaled data \hat{σ}ᵢ_{ⱼ}^{-1/2}Y_{j,k,l} and apply the various methods as before.

The systems and methods described herein may be implemented on a computer system configured for analyzing statistical data, wherein the computer system executes software or firmware instructions to perform steps characteristic of the BAM systems and methods disclosed herein. In one embodiment, the computer system includes a database on a computer-readable storage medium comprising the data. Additionally, or alternatively, the computer system may include a computer-readable storage medium comprising instructions for causing a computer apparatus to execute the steps of the BAM statistical analysis methods described herein. In one embodiment, a network server provides access to instructions for causing a computer system to execute the steps associated with the BAM methods described herein. The server may distribute data fed into, or processed out of the BAM techniques, across a data network.

The contents of all references, patents and published patent applications cited throughout this Application, as well as their associated figures are hereby incorporated by reference in entirety.

Many equivalents to the specific embodiments and the aspects and practices associated with the systems and methods described herein exist. Accordingly, it will be understood that the invention is not to be limited to the embodiments, methods, and practices disclosed herein, but is to be understood from the following claims, which are to be interpreted as broadly as allowed under the law.

1. A method of identifying differentially-expressed genes, comprising:
(a) deriving an analysis of variance (ANOVA) or analysis of covariance (ANCOVA) model for expression data associated with a plurality of genes;
(b) from the ANOVA or ANCOVA model, deriving a linear regression model defined at least in part by an observation vector representative of an observed subset of the gene-expression data, a design matrix of regressor variables, a vector of regression coefficients representing gene contribution to the observation vector, and a measurement error vector; and
(c) to the linear regression model, applying a hierarchical selection algorithm to designate a subset of the regression coefficients as significant regression coefficients, the selection algorithm representing at least one of the observation vector, the design matrix, and the measurement error vector as being hierarchically dependent on parameters having predetermined probabilistic properties, wherein the designated subset corresponds to a respective subset of the genes identified as differentially expressed.

2. The method of claim 1, wherein the parameters are chosen to distinguish statistical properties of the significant regression coefficients.

3. The method of claim 1, wherein the observation vector is at least in part expressed as a sum of:
(a) a vector obtained by multiplicatively applying the design matrix to the regression coefficient vector; and
(b) the measurement error vector.

4. The method of claim 1, wherein the selection algorithm includes a Bayesian selection algorithm.

5. The method of claim 4, wherein the Bayesian selection algorithm uses a posterior distribution of the regression coefficient vector, conditioned on the observation vector, to identify the significant subset of the regression coefficients.

6. The method of claim 5, wherein the Bayesian selection algorithm uses a posterior median of the regression coefficient vector, conditioned on the observation vector, to identify the significant subset of the regression coefficients.

7. The method of claim 5, wherein a posterior variance of the regression coefficient vector, conditioned on the observation vector, is plotted against a posterior mean of the regression coefficient vector, conditioned on the observation vector, to graphically estimate a threshold of comparison parameter used by the selection algorithm.

8. The method of claim 5, wherein the Bayesian selection algorithm uses a posterior mean of the regression coefficient vector, conditioned on the observation vector, to identify the subset of the regression coefficients that are significant.

9. The method of claim 8, wherein the posterior mean is estimated at least in part by using Gibbs sampling.

10. The method of claims 8, wherein the posterior mean of the regression coefficient vector includes a ridge estimate from a generalized ridge regression of the observation vector on the design matrix.

11. The method of claim 10, wherein the posterior mean of the regression coefficient vector includes a weighted average of shrunken posterior estimates of the regression coefficient vector.

12. The method of claims 8, wherein an absolute value of the posterior mean of at least a portion of the regression coefficient vector is entry-wise compared to an entry-wise predetermined threshold value to designate the significant regression coefficients, a regression coefficient being significant if the absolute value of its posterior mean is equal to or exceeds the respective predetermined coefficient.

13. The method of claim 12, wherein the predetermined threshold value is associated with a theoretical limiting variance vector corresponding to entry-wise variances of the conditional mean of the regression coefficient vector.

14. The method of claim 8, wherein, under a null hypothesis indicating substantially no differential gene effect, the distribution of the posterior mean vector is approximated by a two-point mixture probability density.

15. The method of claim 14, wherein the two-point probability density includes a sum of:
(a) a first normal probability density of zero mean and a first variance, the first normal probability density being scaled by a first probability value; and
(b) a second normal probability density of zero mean and a second variance, the second normal probability density being scaled by a second probability value, the second probability value being equal to 1 minus the first probability value.

16. The method of claim 15, wherein the first variance and the second variance are distinct, and one of the first variance and the second variance corresponds to the significant regression coefficients.

17. The method of claim 1, wherein the selection algorithm includes a parametric stochastic variable selection (P-SVS) algorithm.

18. The method of claim 17, wherein a mean of the observation vector is subtracted from the observation vector to create a substantially zero-mean observation vector.

19. The method of claim 18, including, prior to applying the selection algorithm, causing the posterior variance of the regression coefficient vector to be one of a pre-determined set of values by:
(a) an appropriate scaling of the observation vector;
(b) transforming the design matrix to cause each column of the design matrix to have a respective appropriate squared sum value; and
(c) rescaling at least one column of the design matrix by an appropriate design scaling value.

20. The method of claim 19, including a weighted regression step wherein a subset of the observation vector is re-weighted by a standard deviation inverse associated with the subset of the observation vector.

21. The method of claim 19, wherein the pre-determined set is associated with distinct values, each of the distinct values corresponding to a respective hypothesis about the data.

22. The method of claim 19, wherein the value of the appropriate scaling includes a square root of the ratio: size of the observation vector divided by a measurement error variance.

23. The method of claim 19, wherein the appropriate squared sum value includes the observation vector size.

24. The method of claim 19, wherein the appropriate design scaling value causes the columns of the design matrix to have a predetermined second moment.

25. The method of claim 24, wherein the predetermined second moment is about one.

26. A computer apparatus for analyzing statistical data, comprising a set of instructions for causing the apparatus to execute the method of claim 1.

27. The apparatus of claim 26, further comprising a database on a computer-readable storage medium comprising the data.

28. A computer-readable storage medium comprising instructions for causing a computer apparatus to execute the method of claim 1.

29. A network server providing access to instructions for causing a computer apparatus to execute the method of claim 1.

30. The server of claim 29, wherein the server distributes the instructions across a data network.

31. A method of analyzing statistical data, comprising:
(a) deriving an analysis of variance (ANOVA) or analysis of covariance (ANCOVA) model for the data;
(b) from the ANOVA or ANCOVA model, deriving a linear regression model defined at least in part by an observation vector representing the data, a design matrix of regressor variables, a regression coefficient vector, and a measurement error vector; and
(c) to the linear regression model, applying a hierarchical selection algorithm to designate a subset of the regression coefficients as significant regression coefficients, the selection algorithm representing at least one of the observation vector, the design matrix, and the measurement error vector as being hierarchically dependent on a plurality of parameters that have predetermined probabilistic properties.

32. The method of claim 31, wherein the statistical data comprises biomolecular data.

33. The method of claim 32, wherein the biomolecular data comprises biomolecular array data.

34. The method of claim 33, wherein the biomolecular array data is selected from the group consisting of: gene expression data, protein expression data, metabolite production data, genotype data, tissue data, viral data, and a combination thereof.

35. A method of identifying differentially-produced biomolecules, comprising:
(a) deriving an analysis of variance (ANOVA) or analysis of covariance (ANCOVA) model for biomolecular production data associated with a plurality of biomolecules;
(b) from the ANOVA or ANCOVA model, deriving a linear regression model defined at least in part by an observation vector representing the data, a design matrix of regressor variables, a regression coefficient vector, and a measurement error vector; and
(c) to the linear regression model, applying a hierarchical selection algorithm to designate a subset of the regression coefficients as significant regression coefficients, the selection algorithm representing at least one of the observation vector, the design matrix, and the measurement error vector as being hierarchically dependent on a plurality of parameters that have predetermined probabilistic properties.

36. A method of mass-spectroscopic data, comprising:
(a) deriving an analysis of variance (ANOVA) or analysis of covariance (ANCOVA) model for mass-spectroscopic data;
(b) from the ANOVA or ANCOVA model, deriving a linear regression model defined at least in part by an observation vector representing the data, a design matrix of regressor variables, a regression coefficient vector, and a measurement error vector; and
(c) to the linear regression model, applying a hierarchical selection algorithm to designate a subset of the regression coefficients as significant regression coefficients, the selection algorithm representing at least one of the observation vector, the design matrix, and the measurement error vector as being hierarchically dependent on a plurality of parameters that have predetermined probabilistic properties.

37. A method of analyzing statistical data, comprising:
(a) deriving an M-way analysis of variance (ANOVA) or analysis of covariance (ANCOVA) model for the data, M being at least one;
(b) from the ANOVA or ANCOVA model, deriving a linear regression model defined by an observation vector representing the data, a design matrix of regressor variables, a regression coefficient vector, and a measurement error vector; and
(c) to the linear regression model, applying a hierarchical selection algorithm to classify each of the regression coefficients into a number of groups, the selection algorithm representing at least one of the observation vector, the design matrix, and the measurement error vector as being hierarchically dependent on a plurality of parameters that have predetermined probabilistic properties.

38. The method of claim 37, wherein M is at least 3.

39. The method of claim 38, wherein the selection algorithm includes a Bayesian selection algorithm.

40. The method of claim 39, wherein the Bayesian selection algorithm uses a posterior distribution of the regression coefficient vector, conditioned on the observation vector, to identify the significant subset of the regression coefficients.

41. The method of claim 40, including:
(a) designating a first group, from among the M groups, as a baseline reference group; and
(b) plotting Bayes test statistics associated with a second group, distinct from the first group, versus Bayes test statistics associated with the first group.

42. The method of claim 41b, including plotting Bayes test statistics associated with a third group, distinct from each of the first and second groups, versus Bayes test statistics associated with the first group.
